SEQUENCE LISTING (54 sequences)

SEQ ID NO: 1 - protein, 233 aa, Homo sapiens
MDNQGVIYSDLNLPPNPKRQQRKPKGNKSSILATEQEITYAELNLQKASQDFQGNDKTYHCKDLPSAPEKLIVGILGIICLILMASVVTIVVIPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 2 - protein, 288 aa, Homo sapiens
MQIPQAPWPVVWAVLQLGWRPGWFLDSPDRPWNPPTFFPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 3 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 4 - protein, 124 aa, Artificial Sequence
  VH6
EVQLVQSGAEVKKPGESLKISCKGSGYSFTSYWMNWVRQMPGKGLEWMGRIDPYDSETHYSPSFQGQVTISADKSISTAYLQWSSLKASDTAMYYCARGGYDFDVGTLYWFFDVWGQGTTVTVS

SEQ ID NO: 5 - protein, 124 aa, Artificial Sequence
  VH1
QVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVS

SEQ ID NO: 6 - protein, 124 aa, Artificial Sequence
  VH5
EVQLVQSGAEVKKPGESLRISCKGSGYSFTSYWMNWVRQMPGKGLEWMGRIDPYDSETHYSPSFQGHVTISADKSISTAYLQWSSLKASDTAMYYCARGGYDFDVGTLYWFFDVWGQGTTVTVS

SEQ ID NO: 7 - protein, 124 aa, Artificial Sequence
  VH7
EVQLVQSGAEVKKPGATVKISCKVSGYTFTSYWMNWVQQAPGKGLEWMGRIDPYDSETHYAEKFQGRVTITADTSTDTAYMELSSLRSEDTAVYYCATGGYDFDVGTLYWFFDVWGQGTTVTVS

SEQ ID NO: 8 - protein, 124 aa, Artificial Sequence
  VH8
QVQLVQSGAEVKKPGASVKVSCKASGYTFTSYWMNWVRQAPGQGLEWMGRIDPYDSETHYAQKFQGRVTMTRDTSTSTVYMELSSLRSEDTAVYYCARGGYDFDVGTLYWFFDVWGQGTTVTVS

SEQ ID NO: 9 - protein, 107 aa, Artificial Sequence
  Light chain variable region
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIK

SEQ ID NO: 10 - protein, 452 aa, Artificial Sequence
  VH6
EVQLVQSGAEVKKPGESLKISCKGSGYSFTSYWMNWVRQMPGKGLEWMGRIDPYDSETHYSPSFQGQVTISADKSISTAYLQWSSLKASDTAMYYCARGGYDFDVGTLYWFFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 452 aa, Artificial Sequence
  VH1
QVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 452 aa, Artificial Sequence
  VH5
EVQLVQSGAEVKKPGESLRISCKGSGYSFTSYWMNWVRQMPGKGLEWMGRIDPYDSETHYSPSFQGHVTISADKSISTAYLQWSSLKASDTAMYYCARGGYDFDVGTLYWFFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 452 aa, Artificial Sequence
  VH7
EVQLVQSGAEVKKPGATVKISCKVSGYTFTSYWMNWVQQAPGKGLEWMGRIDPYDSETHYAEKFQGRVTITADTSTDTAYMELSSLRSEDTAVYYCATGGYDFDVGTLYWFFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 452 aa, Artificial Sequence
  VH8
QVQLVQSGAEVKKPGASVKVSCKASGYTFTSYWMNWVRQAPGQGLEWMGRIDPYDSETHYAQKFQGRVTMTRDTSTSTVYMELSSLRSEDTAVYYCARGGYDFDVGTLYWFFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 214 aa, Artificial Sequence
  Light chain
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 5 aa, Artificial Sequence
  H-CDR1
SYWMN

SEQ ID NO: 17 - protein, 17 aa, Artificial Sequence
  H-CDR2
RIDPYDSETHYAQKLQG

SEQ ID NO: 18 - protein, 16 aa, Artificial Sequence
  H-CDR3
GGYDFDVGTLYWFFDV

SEQ ID NO: 19 - protein, 11 aa, Artificial Sequence
  L-CDR1
RASENIYSYLA

SEQ ID NO: 20 - protein, 7 aa, Artificial Sequence
  L-CDR2
NAKTLAE

SEQ ID NO: 21 - protein, 9 aa, Artificial Sequence
  L-CDR3
QHHYGTPRT

SEQ ID NO: 22 - protein, 119 aa, Artificial Sequence
  VH
EVQLVESGGGLVKPGGSLKLSCAASGFTFSSYAMSWVRQSPEKRLEWVAEISSGGSYTYYPDTVTGRFTISRDNAKNTLYLEISSLRSEDTAMYYCTRHGDYPRFFDVWGAGTTVTVSS

SEQ ID NO: 23 - protein, 106 aa, Artificial Sequence
  VL
QIVLTQSPALMSASPGEKVTMTCSASSSVSYIYWYQQKPRSSPKPWIYLTSNLASGVPARFSGSGSGTSYSLTISSMEAEDAATYYCQQWSGNPYTFGGGTKLEIK

SEQ ID NO: 24 - protein, 121 aa, Artificial Sequence
  VH
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 25 - protein, 108 aa, Artificial Sequence
  VL
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 26 - protein, 451 aa, Artificial Sequence
  HC
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 215 aa, Artificial Sequence
  LC
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 10 aa, Artificial Sequence
  H-CDR1
GFTFSRYWMS

SEQ ID NO: 29 - protein, 17 aa, Artificial Sequence
  H-CDR2
NIKQDGSEKYYVDSVKG

SEQ ID NO: 30 - protein, 12 aa, Artificial Sequence
  H-CDR3
EGGWFGELAFDY

SEQ ID NO: 31 - protein, 12 aa, Artificial Sequence
  L-CDR1
RASQRVSSSYLA

SEQ ID NO: 32 - protein, 7 aa, Artificial Sequence
  L-CDR2
DASSRAT

SEQ ID NO: 33 - protein, 9 aa, Artificial Sequence
  L-CDR3
QQYGSLPWT

SEQ ID NO: 34 - protein, 118 aa, Artificial Sequence [sequence omitted]

SEQ ID NO: 35 - protein, 122 aa, Artificial Sequence
  VH
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTK

SEQ ID NO: 36 - protein, 108 aa, Artificial Sequence
  VL
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 37 - protein, 445 aa, Artificial Sequence
  HC
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKQVSLTCLVKGFYPSDIAVEWESNGQPENYKTTPPVLDSDGSFFLYSKLTVDKSRWQQGNVFSCSVMHEALHNHYTQKSLSLSPG

SEQ ID NO: 38 - protein, 213 aa, Artificial Sequence
  LC
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 453 aa, Artificial Sequence
  HC
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 214 aa, Artificial Sequence
  LC
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 10 aa, Artificial Sequence
  H-CDR1
GYTFTNYGMN

SEQ ID NO: 42 - protein, 17 aa, Artificial Sequence
  H-CDR2
WINTYTGEPTYAADFKR

SEQ ID NO: 43 - protein, 14 aa, Artificial Sequence
  H-CDR3
YPHYYGSSHWYFDV

SEQ ID NO: 44 - protein, 11 aa, Artificial Sequence
  L-CDR1
SASQDISNYLN

SEQ ID NO: 45 - protein, 7 aa, Artificial Sequence
  L-CDR2
FTSSLHS

SEQ ID NO: 46 - protein, 9 aa, Artificial Sequence
  L-CDR3
QQYSTVPWT

SEQ ID NO: 47 - protein, 449 aa, Artificial Sequence
  HC
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 213 aa, Artificial Sequence
  LC
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGA

SEQ ID NO: 49 - protein, 5 aa, Artificial Sequence
  H-CDR1
NYGVH

SEQ ID NO: 50 - protein, 16 aa, Artificial Sequence
  H-CDR2
VIWSGGNTDYNTPFTS

SEQ ID NO: 51 - protein, 11 aa, Artificial Sequence
  H-CDR3
ALTYYDYEFAY

SEQ ID NO: 52 - protein, 11 aa, Artificial Sequence
  L-CDR1
RASQSIGTNIH

SEQ ID NO: 53 - protein, 7 aa, Artificial Sequence
  L-CDR2
YASESIS

SEQ ID NO: 54 - protein, 9 aa, Artificial Sequence
  L-CDR3
QQNNNWPTT

(19) United States
(12) Patent Application Publication
(10) Pub. No.: US 2022/0211847 A1
(43) Pub. Date: Jul. 7, 2022

(54) COMBINATION OF MONALIZUMAB, DURVALUMAB, CHEMOTHERAPY AND BEVACIZUMAB OR CETUXIMAB FOR THE TREATMENT OF COLORECTAL CANCER

(71) Applicants: MEDIMMUNE LIMITED, CAMBRIDGE, CAMBRIDGESHIRE (GB); INNATE PHARMA, MARSEILLE (FR)

(72) Inventors: SHAAD ESSA ABDULLAH, GAITHERSBURG, MD (US); SHAO-CHUN CHANG, GAITHERSBURG, MD (US); DANIEL J. FREEMAN, GAITHERSBURG, MD (US)

(21) Appl. No.: 17/609,002

(22) PCT Filed: May 6, 2020
(86) PCT No.: PCT/GB2020/051106

§ 371 Date: Nov. 5, 2021

Related U.S. Application Data
(60) Provisional application No. 62/906,191, filed on Sep. 26, 2019.
(60) Provisional application No. 62/843,744, filed on May 6, 2019.

(51) Int. Cl. A61K 39/395; A61K 31/519; A61K 31/513; A61K 31/282; A61P 35/00

(52) U.S. Cl. CPC A61K 39/3955; A61K 31/519; A61K 2039/507; A61K 31/282; A61P 35/00; A61K 31/513

(57) ABSTRACT

The disclosure relates to methods and compositions for the treatment of cancer. Specifically, the disclosure relates to methods comprising administering to a subject in need thereof for the treatment of cancer a NKG2A neutralizing agent, a PD-1 neutralizing agent, a chemotherapy agent, and a VEGF neutralizing agent or an EGFR neutralizing agent.

### FIELD OF THE DISCLOSURE

The disclosure relates to methods and compositions for the treatment of cancer. Specifically, the disclosure relates to methods comprising administering to a subject in need thereof for the treatment of cancer a NKG2A neutralizing agent, a PD-1 neutralizing agent, a chemotherapy agent, and a VEGF neutralizing agent or an EGFR neutralizing agent.

### BACKGROUND

Colorectal cancer (CRC) accounts for 10-15% of all cancers and is the leading cause of cancer deaths in the Western world. Standard of care for treatment of metastatic CRC (mCRC) remains the use of cytotoxic agents. More recently, immunotherapeutic agents have been tested in CRC. Le et al. ((2015) N Engl J Med, 372:2509-2520) conducted a phase 2 clinical study in CRC with pembrolizumab, an anti-programmed death 1 immune checkpoint inhibitor, finding that the immune-related objective response rate and immune-related progression free survival rate were 40% (4 of 10 patients) and 78% (7 of 9 patients) for microsatellite instability (MSI) high CRCs and 0% (0 of 18 patients) and 11% (2 of 18 patients) for microsatellite stable/proficient microsatellite stable (MSS) CRCs, respectively. Only 1 of 10 patients with MSI high (MSI-H) CRC experienced disease progression, as compared to 11/18 MSS CRC patients.

Chemotherapeutic agents and/or targeted therapies do not provide sufficient and/or lasting anti-tumor responses patients having non-microsatellite instability high CRC. There is thus a need for improved benefit to patients treated without DNA repair deficiencies.

### SUMMARY

Provided herein is a method of reducing or inhibiting tumor growth in a subject in need thereof, comprising administering to the subject a therapeutically effective amount of each of a NKG2A neutralizing agent, a PD-1 neutralizing agent, a chemotherapy agent, and a VEGF neutralizing agent.

Further provided herein is a method of treating cancer, in particular colorectal cancer, in a subject in need thereof, comprising administering to the subject a therapeutically effective amount of each of a NKG2A neutralizing agent, a PD-1 neutralizing agent, a chemotherapy agent, and a VEGF neutralizing agent.

Further provided herein is a pharmaceutical formulation comprising a therapeutically effective amount of a NKG2A neutralizing agent, a PD-1 neutralizing agent, a chemotherapy agent, and a VEGF neutralizing agent, for use in treating a subject who has a cancer, in particular a colorectal cancer, wherein the subject has a tumor that is not MSI-H and/or not DNA mismatch-repair (MMR) defective.

Provided herein is a method of reducing or inhibiting tumor growth in a subject in need thereof, comprising administering to the subject a therapeutically effective amount of each of a NKG2A neutralizing agent, a PD-1 neutralizing agent, a chemotherapy agent, and an EGFR neutralizing agent.

Further provided herein is a method of treating cancer, in particular colorectal cancer, in a subject in need thereof, comprising administering to the subject a therapeutically effective amount of each of a NKG2A neutralizing agent, a PD-1 neutralizing agent, a chemotherapy agent, and an EGFR neutralizing agent.

Further provided herein is a pharmaceutical formulation comprising a therapeutically effective amount of a NKG2A neutralizing agent, a PD-1 neutralizing agent, a chemotherapy agent, and an EGFR neutralizing agent, for use in treating a subject who has a cancer, in particular a colorectal cancer, wherein the subject has a tumor that is not MSI-H and/or not DNA mismatch-repair (MMR) defective.

### BRIEF DESCRIPTION OF THE DRAWINGS

FIG. 1 illustrates the percent change in tumor size from baseline and duration of treatment in microsatellite stable (MSS)-CRC expansion cohort that received durvalumab in combination with monalizumab.

FIG. 2 illustrates the percent change in tumor size from baseline and duration of treatment in MSS-CRC patients that received monalizumab, durvalumab, FOLFOX (comprising folinic acid, fluorouracil, and oxaliplatin), and bevacizumab.

FIG. 3A and FIG. 3B illustrate circulating quantities of proliferating (Ki67+) NK and T cell populations assessed using an analytically-validated flow cytometry assay on fresh whole blood specimens from MSS-CRC subjects receiving monalizumab+durvalumab (FIG. 3A) or subjects receiving FOLFOX+bevacizumab+monalizumab+durvalumab (FIG. 3B).

FIG. 4A and FIG. 4B illustrate proliferating CD3+CD4+ and CD8+ T cells and Ki67+ cells in MSS-CRC subjects receiving monalizumab+durvalumab (FIG. 4A) or subjects receiving FOLFOX+bevacizumab+monalizumab and durvalumab (FIG. 4B).

FIG. 5 illustrates the percent change in tumor size from baseline in MSS-CRC patients that received monalizumab, durvalumab, FOLFOX (comprising folinic acid, fluorouracil, and oxaliplatin), and bevacizumab as of 29 Jul. 2019.

FIG. 6 illustrates the percent change in tumor size from baseline and duration of treatment in MSS-CRC patients that received monalizumab, durvalumab, FOLFOX (comprising folinic acid, fluorouracil, and oxaliplatin), and bevacizumab as of 29 Jul. 2019.

FIG. 7 illustrates the percent change in tumor size from baseline and duration of treatment in MSS-CRC patients that received monalizumab, durvalumab, FOLFOX (comprising folinic acid, fluorouracil, and oxaliplatin), and bevacizumab as of 24 Feb. 2020.

FIG. 8 illustrates the percent change in tumor size from baseline in MSS-CRC patients that received monalizumab, durvalumab, FOLFOX (comprising folinic acid, fluorouracil, and oxaliplatin), and bevacizumab as of 24 Feb. 2020.

FIG. 9 illustrates the percent change in tumor size from baseline and duration of treatment in MSS-CRC patients that received monalizumab, durvalumab, FOLFOX (comprising folinic acid, fluorouracil, and oxaliplatin), and cetuximab as of 24 Feb. 2020.

FIG. 10 illustrates the percent change in tumor size from baseline in MSS-CRC patients that received monalizumab, durvalumab, FOLFOX (comprising folinic acid, fluorouracil, and oxaliplatin), and cetuximab as of 24 Feb. 2020.

### DETAILED DESCRIPTION

The disclosure relates to methods and compositions for the treatment of cancer. Specifically, the disclosure relates to methods comprising administering to a subject in need thereof for the treatment of cancer (i) a NKG2A neutralizing agent, a PD-1 neutralizing agent, a chemotherapy agent, and a VEGF neutralizing agent; or (ii) a NKG2A neutralizing agent, a PD-1 neutralizing agent, a chemotherapy agent, and an EGFR neutralizing agent.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

The term “antibody” as used herein refers to a protein that is capable of recognizing and specifically binding to an antigen. Ordinary or conventional mammalian antibodies comprise a tetramer, which is typically composed of two identical pairs of polypeptide chains, each pair consisting of one “light” chain (typically having a molecular weight of about 25 kDa) and one “heavy” chain (typically having a molecular weight of about 50-70 kDa). The terms “heavy chain” and “light chain” as used herein, refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The carboxyl-terminal portion of each chain typically defines a constant domain responsible for effector function. Thus, in a typical IgG, IgA or IgD naturally occurring antibody, a full-length heavy chain immunoglobulin polypeptide includes a variable domain (V_{H}) and three constant domains (C_{H1}, C_{H2}, and C_{H3}) and a hinge region between C_{H1} and C_{H2}, wherein the V_{H} domain is at the amino-terminus of the polypeptide and the C_{H3} domain is at the carboxyl-terminus, and a full-length light chain immunoglobulin polypeptide includes a variable domain (V_{L}) and a constant domain (CL), wherein the V_{L} domain is at the amino-terminus of the polypeptide and the C_{L} domain is at the carboxyl-terminus. A typical IgM or IgE antibody has a similar structure as mentioned above for an IgG, IgA or IgD, except for the presence of an additional constant domain, C_{H3}, and the absence of a hinge region between C_{H1} and C_{H2}.

Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen-binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

NKG2A (OMIM 161555) is a member of the NKG2 group of transcripts (Houchins, et al. (1991) J. Exp. Med. 173:1017-1020). NKG2A is encoded by 7 exons spanning 25 kb, showing some differential splicing. Together with CD94, NKG2A forms the heterodimeric inhibitory receptor CD94/NKG2A, found on the surface of subsets of NK cells, α/β T cells, γ/δ T cells, and NKT cells. Similar to inhibitory MR receptors, it possesses an ITIM in its cytoplasmic domain. As used herein, “NKG2A” refers to any variant, derivative, or isoform of the NKG2A gene or encoded protein. Human NKG2A comprises 233 amino acids in 3 domains, with a cytoplasmic domain comprising residues 1-70, a transmembrane region comprising residues 71-93, and an extracellular region comprising residues 94-233, of the following sequence:

(SEQ ID NO: 1)
MDNQGVIYSDLNLPPNPKRQQRKPKGNKSSILATEQEITYAELNLQKASQD
FQGNDKTYHCKDLPSAPEKLIVGILGIICLILMASVVTIVVIPSTLIQRHN
NSSLNTRTQKARHCGHCPEEWITYSNSCYYIGKERRTWEESLLACTSKNSS
LLSIDNEEEMKFLSIISPSSWIGVERNSSHHPWVTMNGLAFKHEIKDSDNA
ELNCAVLQVNRLKSAQCGSSIIYHCKHKL.

NKG2C (OMIM 602891) and NKG2E (OMIM 602892) are two other members of the NKG2 group of transcripts (Gilenke, et al. (1998) Immunogenetics 48:163-173). The CD94/NKG2C and CD94/NKG2E receptors are activating receptors found on the surface of subsets of lymphocytes such as NK cells and T-cells.

HLA-E (OMIM 143010) is a nonclassical MHC molecule that is expressed on the cell surface and regulated by the binding of peptides, e.g. such as fragments derived from the signal sequence of other MHC class I molecules. Soluble versions of HLA-E have also been identified. In addition to its T-cell receptor binding properties, HLA-E binds subsets of natural killer (NK) cells, natural killer T-cells (NKT) and T cells (α/β and γ/δ), by binding specifically to CD94/NKG2A, CD94/NKG2B, and CD94/NKG2C (see, e.g., Braud et al. (1998) Nature 391:795-799, the entire disclosure of which is herein incorporated by reference). Surface expression of HLA-E protects target cells from lysis by CD94/NKG2A+NK, T, or NKT cell clones. As used herein, “HLA-E” refers to any variant, derivative, or isoform of the HLA-E gene or encoded protein.

“Reduces the inhibitory activity of NKG2A”, “neutralizes NKG2A” or “neutralizes the inhibitory activity of NKG2A” refers to a process in which CD94/NKG2A is inhibited in its capacity to negatively affect intracellular processes leading to lymphocyte responses such as cytokine release and cytotoxic responses.

In some embodiments, the NKG2A neutralizing agent binds an extra-cellular portion of human CD94/NKG2A receptor or its ligand HLA-E and reduces the inhibitory activity of human CD94/NKG2A receptor expressed on the surface of a CD94/NKG2A positive lymphocyte. In some embodiments the agent competes with HLA-E in binding to CD94/NKG2A, i.e. the agent blocks the interaction between CD94/NKG2A and its ligand HLA-E. In another embodiment the agent binds NKG2A but does not compete with HLA-E in binding to CD94/NKG2A; i.e. the agent is capable of binding CD94/NKG2A simultaneously with HLA-E.

In some embodiments, the NKG2A neutralizing agent is an antibody or an antigen-binding fragment thereof that binds a human NKG2A protein. In some embodiments, the antibody or an antigen-binding fragment thereof is a humanized or human anti-NKG2A antibody. In some embodiments, the NKG2A neutralizing agent is an antibody or an antigen-binding fragment thereof that inhibits binding of NKG2A to HLA-E.

In some embodiments, the NKG2A neutralizing agent is an antibody selected from a fully human antibody, a humanized antibody, and a chimeric antibody. In some embodiments, the agent comprises a constant domain derived from a human IgG1, IgG2, IgG3 or IgG4 antibody. In some embodiments, the agent is a fragment of an antibody selected from IgA, an IgD, an IgG, an IgE and an IgM antibody. In some embodiments, the agent is an antibody fragment selected from a Fab fragment, a Fab′ fragment, a Fab′-SH fragment, a F(ab)2 fragment, a F(ab′)2 fragment, an Fv fragment, a Heavy chain Ig (a llama or camel Ig), a VHH fragment, a single domain FV, and a single-chain antibody fragment. In some embodiments, the agent is a synthetic or semisynthetic antibody-derived molecule selected from a scFV, a dsFV, a minibody, a diabody, a triabody, a kappa body, an IgNAR, and a multispecific antibody.

In some embodiments, the anti-NKG2A antibodies do not demonstrate substantial specific binding to human Fcγ receptors, e.g. CD16. In some embodiments, the anti-NKG2A antibodies lack substantial specific binding or have low or decreased specific binding to one or more, or all of, human CD16, CD32A, CD32B or CD64. Exemplary antibodies may comprise constant regions of various heavy chains that are known not to bind or to have low binding to Fcγ receptors. One such example is a human IgG4 constant region. In some embodiments, the IgG4 antibody comprises a modification to prevent the formation of half antibodies (fab arm exchange) in vivo, e.g., the antibody comprises an IgG4 heavy chain comprising a serine to proline mutation in residue 241, corresponding to position 228 according to the EU-index (Kabat et al., “Sequences of proteins of immunological interest”, 5^{th} ed., NIH, Bethesda, M L, 1991). Such modified IgG4 antibodies will remain intact in vivo and maintain a bivalent (high affinity) binding to NKG2A, as opposed to native IgG4 that will undergo fab arm exchange in vivo such that they bind to NKG2A in monovalent manner which can alter binding affinity. Alternatively, antibody fragments that do not comprise constant regions, such as Fab or F(ab′)2 fragments, can be used to avoid Fc receptor binding. Fc receptor binding can be assessed according to methods known in the art, including for example testing binding of an antibody to Fc receptor protein in a BIACORE assay. Also, any human antibody type (e.g. IgG1, IgG2, IgG3 or IgG4) can be used in which the Fc portion is modified to minimize or eliminate binding to Fc receptors (see, e.g., WO03101485, the disclosure of which is herein incorporated by reference). Assays such as, e.g., cell based assays, to assess Fc receptor binding are well known in the art, and are described in, e.g., WO03101485.

The anti-NKG2A antibody can be a humanized antibody, for example comprising a VH human acceptor framework from a human acceptor sequence selected from, e.g., VH1_18, VH5_a, VH5_51, VH1_f, and VH1_46, and a JH6 J-segment, or other human germline VH framework sequences known in the art. The VL region human acceptor sequence may be, e.g., VKI_O2/JK4.

In some embodiments, the antibody is a humanized antibody based on antibody Z270. Different humanized Z270 heavy chain variable regions are shown in SEQ ID NOS: 4-8, and can further comprise a C-terminal serine (S) residue. The HumZ270VH6 variable region of SEQ ID NO: 4 is based on a human VH5_51 gene; the HumZ270VH1 variable region of SEQ ID NO: 5 is based on a human VH1_18 gene; the humZ270VH5 variable region of SEQ ID NO: 6 is based on a human VH5_a gene; the humZ270VH7 variable region of SEQ ID NO: 7 is based on a human VH1_f gene; and the humZ270VH8 variable region of SEQ ID NO: 8 is based on a human VH1_46 gene; all with a human JH6 J-segment. Each of these antibodies retains high affinity binding to NKG2A, with low likelihood of a host immune response against the antibody as the 6 C-terminal amino acid residues of the Kabat H-CDR2 of each of the humanized constructs are identical to the human acceptor framework. Using the alignment program VectorNTI, the following sequence identities between humZ270VH1 and humZ270VH5, -6, -7, and -8 were obtained: 78.2% (VH1 vs. VH5), 79.0% (VH1 vs. VH6), 88.7% (VH1 vs. VH7), and 96.0% (VH1 vs. VH8).

In some embodiments, the NKG2A neutralizing agent comprises (i) a heavy chain variable region of SEQ ID NOS: 4-8, or an amino acid sequence at least 50%, 60%, 70%, 80%, 90%, 95%, 98% or 99% identical thereto, and (ii) a light chain variable region of SEQ ID NO: 9, or an amino acid sequence at least 50%, 60%, 70%, 80%, 90%, 95%, 98% or 99% identical thereto. In some embodiments, the agent comprises (i) a heavy chain comprising the amino acid sequence of any of SEQ ID NOS: 10-14, or an amino acid sequence at least 50%, 60%, 70%, 80%, 90%, 95%, 98% or 99% identical thereto, and (ii) a light chain comprising the amino acid sequence of SEQ ID NO: 15, or an amino acid sequence at least 50%, 60%, 70%, 80%, 90%, 95%, 98% or 99% identical thereto.

In some embodiments, the antibody comprising a heavy chain variable region of any of SEQ ID NOS: 4-8 and a light chain variable region comprising SEQ ID NO: 9 neutralizes the inhibitory activity of NKG2A, but does not substantially bind the activating receptors NKG2C, NKG2E or NKG2H. The antibody can furthermore compete with HLA-E for binding to NKG2A on the surface of a cell. In some embodiments, the agent comprises H-CDR1, H-CDR2 and/or H-CDR3 sequences derived from the heavy chain variable region having the amino acid sequence of any of SEQ ID NOS: 4-8. In some embodiments, the agent comprises L-CDR1, L-CDR2 and/or L-CDR3 sequences derived from the light chain variable region having the amino acid sequence of SEQ ID NO: 9.

Heavy chain variable regions
VH6:
(SEQ ID NO: 4)
EVQLVQSGAEVKKPGESLKISCKGSGYSFTSYWMNWVRQMPGKGLEWMGRI
DPYDSETHYSPSFQGQVTISADKSISTAYLQWSSLKASDTAMYYCARGGYD
FDVGTLYWFFDVWGQGTTVTVS
VH1:
(SEQ ID NO: 5)
QVQLVQSGAEVKKPGASVKVSCKASGYTFTSYWMNWVRQAPGQGLEWMGRI
DPYDSETHYAQKLQGRVTMTTDTSTSTAYMELRSLRSDDTAVYYCARGGYD
FDVGTLYWFFDVWGQGTTVTVS
VH5:
(SEQ ID NO: 6)
EVQLVQSGAEVKKPGESLRISCKGSGYSFTSYWMNWVRQMPGKGLEWMGRI
DPYDSETHYSPSFQGHVTISADKSISTAYLQWSSLKASDTAMYYCARGGYD
EDVGTLYWFFDVWGQGTTVTVS
VH7:
(SEQ ID NO: 7)
EVQLVQSGAEVKKPGATVKISCKVSGYTFTSYWMNWVQQAPGKGLEWMGRI
DPYDSETHYAEKFQGRVTITADTSTDTAYMELSSLRSEDTAVYYCATGGYD
FDVGTLYWFFDVWGQGTTVTVS
VH8:
(SEQ ID NO: 8)
QVQLVQSGAEVKKPGASVKVSCKASGYTFTSYWMNWVRQAPGQGLEWMGRI
DPYDSETHYAQKFQGRVTMTRDTSTSTVYMELSSLRSEDTAVYYCARGGYD
FDVGTLYWFFDVWGQGTTVTVS
Light chain variable region
(SEQ ID NO: 9)
DIQMTQSPSSLSASVGDRVTITCRASENIYSYLAWYQQKPGKAPKLLIYNA
KTLAEGVPSRFSGS GSGTDFTLTISSLQPEDFATYYCQHHYGTPRTFGGG
TKVEIK
Heavy Chains (variable region domain amino acids
underlined)
VH6:
(SEQ ID NO: 10)
EVQLVQSGAEVKKPGESLKISCKGSGYSFTSYWMNWVRQMPGKGLEWMGRI
DPYDSETHYSPSFQGQVTISADKSISTAYLQWSSLKASDTAMYYCARGGYD
FDVGTLYWFFDVWGQGTTVTVSSASTKGPSVFPLAPCSRSTSESTAALGCL
VKDYFPEPVTVSWNSGALTSGVHTFPAVLQSSGLYSLSSVVTVPSSSLGTK
TYTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK
VH1:
(SEQ ID NO: 11)
QVQLVQSGAEVKKPGASVKVSCKASGYTFTSYWMNWVRQAPGQGLEWMGRI
DPYDSETHYAQKLQGRVTMTTDTSTSTAYMELRSLRSDDTAVYYCARGGYD
FDVGTLYWFFDVWGQGTTVTVSSASTKGPSVFPLAPCSRSTSESTAALGCL
VKDYFPEPVTVSWNSGALTSGVHTEPAVLQSSGLYSLSSVVTVPSSSLGTK
TYTCNVDHKPSNTKVDKRVESKYGPPCPPCPAPEELGGPSVFLFPPKPKDT
LMISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDGS
FFLYSRLTVDKSRWQEGNVFSCSVMHEALHNHYTQKSLS LSLGK
VH5:
(SEQ ID NO: 12)
EVQLVQSGAEVKKPGESLRISCKGSGYSFTSYWMNWVRQMPGKGLEWMGRI
DPYDSETHYSPSFQGHVTISADKSISTAYLQWSSLKASDTAMYYCARGGYD
FDVGTLYWFFDVWGQGTTVTVSSASTKGPSVFPLAPCSRSTSESTAALGCL
VKDYFPEPVTVSWNSGALTSGVHTEPAVLQSSGLYSLSSVVTVPSSSLGTK
TYTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTLP
PSQEEMTKNQVSLTCLVKGFYSDIAVEWSNGQPENNYKTTPPVLDSDGSFE
LYSRLTVDKSRWQEGNVFSCSVMHEALHNHYTQKSLS LSLGK
VH7:
(SEQ ID NO: 13)
EVQLVQSGAEVKKPGATVKISCKVSGYTFTSYWMNWVQQAPGKGLEWMGRI
DPYDSETHYAEKFQGRVTITADTSTDTAYMELSSLRSEDTAVYYCATGGYD
FDVGTLYWFFDVWGQGTTVTVSSASTKGPSVFPLAPCSRSTSESTAALGCL
VKDYFPEPVTVSWNSGALTSGVHTFPAVLQSSGLYSLSSVVTVPSSSLGTK
TYTCNVDHKPSNTKVDKRVESKYGPPCPPCPAPEFLGGPSVFLFPPKPKDT
LMISRTPEVTCVVVDVSQEDPEVQENWYVDGVEVHNAKTKPREEQFNSTYR
VVSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDGS
FFLYSRLTVDKSRWQEGNVFSCSVMHEALHNHYTQKSLS LSLGK
VH8:
(SEQ ID NO: 14)
QVQLVQSGAEVKKPGASVKVSCKASGYTFTSYWMNWVRQAPGQGLEWMGRI
DPYDSETHYAQKFQGRVTMTRDTSTSTVYMELSSLRSEDTAVYYCARGGYD
FDVGTLYWFFDVWGQGTTVTVSSASTKGPSVFPLAPCSRSTSESTAALGCL
VKDYFPEPVTVSWNSGALTSGVHTFPAVLQSSGLYSLSSVVTVPSSSLGTK
TYTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK
Light chain (variable region domain amino acids
underlined)
(SEQ ID NO: 15)
DIQMTQSPSSLSASVGDRVTITCRASENIYSYLAWYQQKPGKAPKLLIYNA
KTLAEGVPSRFSGSGSGTDFTLTISSLQPEDFATYYCQHHYGTPRTFGGGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC
Monalizumab, CDRs on heavy and light chains
Heavy chain CDRs, according to Kabat numbering
scheme:
H-CDR1:
(SEQ ID NO: 16)
SYWMN
H-CDR2:
(SEQ ID NO: 17)
RIDPYDSETHYAQKLQG
H-CDR3:
(SEQ ID NO: 18)
GGYDFDVGTLYWFFDV
Light chain CDRs according to Kabat numbering
scheme:
L-CDR1:
(SEQ ID NO: 19)
RASENIYSYLA
L-CDR2:
(SEQ ID NO: 20)
NAKTLAE
L-CDR3:
(SEQ ID NO: 21)
QHHYGTPRT

In some embodiments, the anti-NKG2A antibody is an antibody comprising a H-CDR1 corresponding to residues 31-35 of SEQ ID NOS: 4-8 (or of SEQ ID NOS: 10-14), a H-CDR2 corresponding to residues 50-60 (optionally 50-66 when including amino acids of human origin) of SEQ ID NOS: 4-8 (or of SEQ ID NOS: 10-14), and a H-CDR3 corresponding to residues 99-114 (95-102 according to Kabat) of SEQ ID NOS: 4-8 (or of SEQ ID NOS: 10-14). In some embodiments, the H-CDR2 corresponding to residues 50-66 of SEQ ID NOS: 4-8 (or of SEQ ID NOS: 10-14). In some embodiments, a CDR comprises one, two, three, four, or more amino acid substitutions.

In some embodiments, the anti-NKG2A antibody is an antibody comprising a L-CDR1 corresponding to residues 24-34 of SEQ ID NOS: 9 or 15, a L-CDR2 corresponding to residues 50-56 of SEQ ID NOS: 9 or 15, and an L-CDR3 corresponding to residues 89-97 of SEQ ID NOS: 9 or 15. Optionally, a CDR may comprise one, two, three, four, or more amino acid substitutions.

In some embodiments, the anti-NKG2A antibody is an antibody comprising a H-CDR1 corresponding to residues 31-35 of SEQ ID NOS: 4-8, a H-CDR2 corresponding to residues 50-60 (optionally 50-66) of SEQ ID NOS: 4-8, and a H-CDR3 corresponding to residues 99-114 (95-102 according to Kabat) of SEQ ID NOS: 4-8, a L-CDR1 corresponding to residues 24-34 of SEQ ID NO: 9, a L-CDR2 corresponding to residues 50-56 of SEQ ID NO: 9, and an L-CDR3 corresponding to residues 89-97 of SEQ ID NO: 9.

In some embodiments, the anti-NKG2A antibody is an antibody comprising the heavy chain H-CDR1, H-CDR2 and H-CDR3 domains having the amino acid sequences of SEQ ID NOS: 16-18, and the light chain L-CDR1, L-CDR2 and L-CDR3 domains having the amino acid sequences of SEQ ID NOS: 19-21, respectively.

In some embodiments, the NKG2A-neutralizing agent is monalizumab, an anti-NKG2A antibody having the heavy chain variable region amino acid sequence of SEQ ID NO: 5 and the light chain variable region amino acid sequence of SEQ ID NO: 9. In some embodiments, the agent is monalizumab, an anti-NKG2A antibody having the heavy chain amino acid sequence of SEQ ID NO: 11 and the light chain amino acid sequence of SEQ ID NO: 15.

In some embodiments, the NKG2A-neutralizing agent comprises H-CDR1, H-CDR2 and/or H-CDR3 sequences derived from the VH having the amino acid sequence of SEQ ID NO: 22. In some embodiments, the agent comprises L-CDR1, L-CDR2 and/or L-CDR3 sequences derived from the VL having the amino acid sequence of SEQ ID NO: 23. In some embodiments, the agent comprises H-CDR1, H-CDR2 and/or H-CDR3 sequences derived from the VH having the amino acid sequence of SEQ ID NO: 22, and L-CDR1, L-CDR2 and/or L-CDR3 sequences derived from the VL having the amino acid sequence of SEQ ID NO: 23. The antibody having the heavy chain variable region of SEQ ID NO: 22 and a light chain variable region of SEQ ID NO: 23 neutralizes the inhibitory activity of NKG2A, and also binds the activating receptors NKG2C, NKG2E or NKG2H. This antibody does not compete with HLA-E for binding to NKG2A on the surface of a cell (i.e. it is a non-competitive antagonist of NKG2A).

(SEQ ID NO: 22)
EVQLVESGGGLVKPGGSLKLSCAASGfTFSSYAMSWVRQSPEKRLEWVAEI
SSGGSYTYYPDTVTGRFTISRDNAKNTLYLEISSLRSEDTAMYYCTRHGDY
PRFFDVWGAGTTVTVSS
(SEQ ID NO: 23)
QIVLTQSPALMSASPGEKVTMTCSASSSVSYIYwYQQKPRSSPKPWIYLTS
NLASGVPARFSGSGSGTSYSLTISSMEAEDAATYYCQQWSGNPYTfGGGTK
LEIK

In some embodiments, the NKG2A neutralizing agent comprises amino acid residues 31-35, 50-60, 62, 64, 66, and 99-108 of the variable-heavy (VH) domain (SEQ ID NO: 22 and amino acid residues 24-33, 49-55, and 88-96 of the variable-light (VL) domain (SEQ ID NO: 23), optionally with one, two, three, four, or more amino acid substitutions. In some embodiments, the NKG2A neutralizing agent is a humanized antibody, for example an agent comprising heavy and light chain variable regions as disclosed in PCT publication no. WO2009/092805, the disclosure of which is incorporated herein by reference.

In some embodiments, the NKG2A neutralizing agent is a fully human antibody which has been raised against the CD94/NKG2A epitope to which any of the aforementioned antibodies bind.

It will be appreciated that, while the aforementioned antibodies can be used, other antibodies can recognize and be raised against any part of the NKG2A polypeptide so long as the antibody causes the neutralization of the inhibitory activity of NKG2A. For example, any fragment of NKG2A, including NKG2A, or any combination of NKG2A fragments, can be used as immunogens to raise antibodies, and the antibodies can recognize epitopes at any location within the NKG2A polypeptide, so long as they can do so on NKG2A expressing NK cells as described herein. In some embodiments, the epitope is the epitope specifically recognized by an antibody having a heavy chain variable region of SEQ ID NOS: 4-8 and a light chain variable region of SEQ ID NO: 9.

In some embodiments, the NKG2A neutralizing agent competes with humZ270 antibody disclosed in U.S. Pat. No. 8,206,709 (the disclosure of which is incorporated herein by reference) in binding to the extra-cellular portion of human CD94/NKG2A receptor. Competitive binding can be measured, for instance, in BiaCore experiments, in which the capacity of agents is measured, for binding the extracellular portion of immobilized CD94/NKG2A receptor (e.g. purified from CD94/NKG2 expressing cells, or produced in a bio-system) saturated with humZ270. Alternatively, the binding of agents to cells is measured that either naturally express, or over-express (e.g. after transient or stable transfection), CD94/NKG2A receptor, and which have been pre-incubated with saturating doses of Z270. In one embodiment, competitive binding can be measured using the methods disclosed in U.S. Pat. No. 8,206,709, for example by assessing binding to Ba/F3-CD94-NKG2A cells by flow cytometry as shown in Example 15 of U.S. Pat. No. 8,206,709, the disclosure of which is incorporated herein by reference.

As used herein, the terms “PD-1” refers to the protein Programmed Death 1 (PD-1) (also referred to as “Programmed Cell Death 1”), an inhibitory member of the CD28 family of receptors, that also includes CD28, CTLA-4, ICOS and BTLA. The complete human PD-1 sequence can be found under GenBank Accession No. U64863, shown as follows:

(SEQ ID NO: 2)
MQIPQAPWPVVWAVLQLGWRPGWFLDSPDRPWNPPTFFPALLVVTEGDNAT
FTCSFSNTSESFVLNWYRMSPSNQTDKLAAFPEDRSQPGQDCRFRVTQLPN
GRDFHMSVVRARRNDSGTYLCGAISLAPKAQIKESLRAELRVTERRAEVPT
AHPSPSPRPAGQFQTLVVGVVGGLLGSLVLLVWVLAVICSRAARGTIGARR
TGQPLKEDPSAVPVFSVDYGELDFQWREKTPEPPVPCVPEQTEYATIVFPS
GMGTSSPARRGSADGPRSAQPLRPEDGHCSWPL.

“PD-1” also includes any variant, derivative, or isoform of the PD-1 gene or encoded protein. PD-1 is expressed on activated B cells, T cells, and myeloid cells (Okazaki et al. (2002) Curr. Opin. Immunol. 14: 391779-82; Bennett et al. (2003) J Immunol 170:711-8). The initial members of the family, CD28 and ICOS, were discovered by functional effects on augmenting T cell proliferation following the addition of monoclonal antibodies (Hutloff et al. (1999) Nature 397:263-266; Hansen et al. (1980) Immunogenics 10:247-260). Two ligands for PD-1 have been identified, PD-L1 and PD-L2, that have been shown to downregulate T cell activation upon binding to PD-1 (Freeman et al. (2000) J Exp Med 192:1027-34; Latchman et al. (2001) Nat Immunol 2:261-8; Carter et al. (2002) Eur J Immunol 32:634-43). Both PD-L1 and PD-L2 are B7 homologs that bind to PD-1, but do not bind to other CD28 family members.

The complete human PD-L1 sequence can be found under UniProtKB/Swiss-Prot, identifier Q9NZQ7-1, shown as follows:

(SEQ ID NO: 3)
MRIFAVFIFMTYWHLLNAFTVTVPKDLYVVEYGSNMTIECKFPVEKQLDLA
ALIVYWEMEDKNIIQFVHGEEDLKVQHSSYRQRARLLKDQLSLGNAALQIT
DVKLQDAGVYRCMISYGGADYKRITVKVNAPYNKINQRILVVDPVTSEHEL
TCQAEGYPKAEVIWTSSDHQVLSGKTTTTNSKREEKLFNVTSTLRINTTTN
EIFYCTFRRLDPEENHTAELVIPELPLAHPPNERTHLVILGAILLCLGVAL
TFIFR LRKGRMMDVK KCGIQDTNSKKQSDTHLEET.

PD-L1 is abundant in a variety of human cancers (Dong et al. (2002) Nat. Med. 8:787-9). The interaction between PD-1 and PD-L1 results in a decrease in tumor infiltrating lymphocytes, a decrease in T-cell receptor mediated proliferation, and immune evasion by the cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1, and the effect is additive when the interaction of PD-1 with PD-L2 is blocked as well.

A PD-1 neutralizing agent is an agent that neutralizes PD-1 or reduces the inhibitory activity of human PD-1. “Reduces the inhibitory activity of human PD-1”, “neutralizes PD-1” or “neutralizes the inhibitory activity of human PD-1” refers to a process in which PD-1 is inhibited in its signal transduction capacity resulting from the interaction of PD-1 with one or more of its binding partners, such as PD-L1 or PD-L2. An agent that neutralizes the inhibitory activity of PD-1 decreases, blocks, inhibits, abrogates or interferes with signal transduction resulting from the interaction of PD-1 with one or more of its binding partners, such as PD-L1, PD-L2. Such an agent can thereby reduce the negative co-stimulatory signal mediated by or through cell surface proteins expressed on T lymphocytes, so as to enhance T-cell effector functions such as proliferation, cytokine production and/or cytotoxicity. A PD-1 neutralizing agent can interact with PD-1 and/or with one or more of its binding partners, e.g. PD-L1 and PD-L2.

In some embodiments, the PD-1 neutralizing agent is an antibody or an antigen-binding fragment thereof. In some embodiments, the PD-1 neutralizing agent is an antibody or an antigen-binding fragment thereof that binds a human PD-1 polypeptide. In some embodiments, the PD-1 neutralizing agent is a human anti-PD-L1 antibody or an antigen-binding fragment.

In some embodiments, the PD-1 neutralizing agent is an anti-PD-L1 monoclonal antibody that inhibits the binding of PD-L1 to PD-1. In some embodiments, the PD-1 neutralizing agent is an anti-PD-1 monoclonal antibody that inhibits the binding of PD-1 to PD-L1. In some embodiments, the PD-1 neutralizing agent is an immunoadhesin (e.g., an immunoadhesin comprising an extracellular or PD-1 binding portion of PD-L1 or PD-L2 fused to a constant region (e.g., an Fc region of an immunoglobulin sequence)).

In some embodiments, the PD-1 neutralizing agent is YW243.55.S70, MPDL3280A (atezolizumab, Tecentriq®), MDX-1105, or durvalumab (MEDI4736, Imfinzi®). MDX-1105, also known as BMS-936559, is an anti-PD-L1 antibody described in WO2007/005874. Antibody YW243.55.S70 is an anti-PD-L1 described in WO 2010/077634. Examples of anti-PD-L1 antibodies useful for the methods disclosed herein, and methods for making thereof are also described in WO 2010/077634 A1 and U.S. Pat. No. 8,217,149, which are incorporated herein by reference.

In some embodiments, the PD-1 neutralizing agent is a PD-L1 antibody that is durvalumab. Durvalumab (MEDI4736, Imfinzi™) is a human monoclonal antibody directed against human PD-L1 that is capable of blocking the binding of PD-L1 to both the PD-1 and CD80 receptors. Disclosure related to durvalumab can be found in U.S. Pat. Nos. 8,779,108 and 9,493,565, which are incorporated herein by reference. Durvalumab has the heavy and light chains of amino acid sequences SEQ ID NO: 26 and SEQ ID NO: 27, respectively. The heavy chain variable region of durvalumab is shown in SEQ ID NO: 24 and the light chain variable region of durvalumab is shown in SEQ ID NO: 25.

In another embodiment, the PD-1 neutralizing agent is an anti-PD-L1 antibody (or an antigen-binding portion thereof) competing with durvalumab for binding to PD-L1. In some embodiments, the anti-PD-L1 antibody binds to the same epitope as durvalumab. In certain embodiments, the anti-PD-L1 antibody has the same heavy and light chain CDRs as durvalumab.

In some embodiments, the PD-1 neutralizing agent (e.g. an agent derived from durvalumab) comprises (i) the heavy chain variable region of SEQ ID NO: 24, or an amino acid sequence at least 50%, 60%, 70%, 80%, 90%, 95%, 98% or 99% identical thereto, and (ii) the light chain variable region of SEQ ID NO: 25, or an amino acid sequence at least 50%, 60%, 70%, 80%, 90%, 95%, 98% or 99% identical thereto. In some embodiments, the PD-1 neutralizing agent (e.g. an agent derived from durvalumab) comprises (i) the heavy chain of SEQ ID NO: 26, or an amino acid sequence at least 50%, 60%, 70%, 80%, 90%, 95%, 98% or 99% identical thereto, and (ii) the light chain of SEQ ID NO: 27, or an amino acid sequence at least 50%, 60%, 70%, 80%, 90%, 95%, 98% or 99% identical thereto. In some embodiments, the PD-1 neutralizing agent comprises H-CDR1, H-CDR2 and/or H-CDR3 sequences derived from the heavy chain variable region comprising the amino acid sequence of SEQ ID NO: 24. In some embodiments, the PD-1 neutralizing agent comprises L-CDR1, L-CDR2 and/or L-CDR3 sequences derived from the light chain variable region comprising the amino acid sequence of SEQ ID NO: 25.

In some embodiments, the PD-1 neutralizing agent comprises the heavy chain H-CDR1, H-CDR2 and H-CDR3 domains having the amino acid sequences of SEQ ID NOS: 28-30, respectively, and the light chain L-CDR1, L-CDR2, L-CDR3 domains having the amino acid sequences of SEQ ID NOS: 31-33, respectively.

Heavy chain variable region of Durvalumab:
(SEQ ID NO: 24)
EVQLVESGGGLVQPGGSLRLSCAASGFTFSRYWMSWVRQAPGKGLEWVANI
KQDGSEKYYVDSVKGRFTISRDNAKNSLYLQMNSLRAEDTAVYYCAREGGW
FGELAFDYWGQGTLVTVSS
Light chain variable region of Durvalumab
(SEQ ID NO: 25)
EIVLTQSPGTLSLSPGERATLSCRASQRVSSSYLAWYQQKPGQAPRLLIYD
ASSRATGIPDRFSGSGS GTDFTLTISRLEPEDFAVYYCQQYGSLPWTFGQ
GTKVEIK
Heavy chain of Durvalumab (variable region
underlined)
(SEQ ID NO: 26)
EVQLVESGGGLVQPGGSLRLSCAASGFTFSRYWMSWVRQAPGKGLEWVANI
KQDGSEKYYVDSVKGRFTISRDNAKNSLYLQMNSLRAEDTAVYYCAREGGW
FGELAFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYIC
NVNHKPSNTKVDKRVEPKSCDKTHTCPPCPAPEFEGGPSVFLFPPKPKDTL
MISRTPEVTCVVVDVSHEDPEVKFNWVYVDGVEVHNAKTKPREEQYNSTYR
VVSVLTVLHQDWLNGKEYKCKVSNKALPASIEKTISKAKGQPREPQVYTLP
PSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
Light chain of Durvalumab (variable region
underlined)
(SEQ ID NO: 27)
EIVLTQSPGTLSLSPGERATLSCRASQRVSSSYLAWYQQKPGQAPRLLIYD
ASSRATGIPDRFSGSGSGTDFTLTISRLEPEDFAVYYCQQYGSLPWTFGQG
TKVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGL S
SPVTKSFNRGEC
Durvalumab, Heavy chain CDRs:
H-CDR1:
(SEQ ID NO: 28)
GFTFSRYVVMS
H-CDR2:
(SEQ ID NO: 29)
NIKQDGSEKYYVDSVKG
H-CDR3:
SEQ ID NO: 30)
EGGWFGELAFDY
Durvalumab, Light chain CDRs:
L-CDR1:
(SEQ ID NO: 31)
RASQRVSSSYLA
L-CDR2:
(SEQ ID NO: 32)
DASSRAT
L-CDR3:
(SEQ ID NO: 33)
QQYGSLPWT

In another embodiment, the PD-1 neutralizing agent is an anti-PD-L1 antibody that is atezolizumab (MPDL3280A, Tecentriq®, CAS Registry Number: 1422185-06-5). In some embodiments, the anti-PD-L1 antibody comprises a heavy chain variable region comprising the amino acid sequence:

(SEQ ID NO: 34)
EVQLVESGGGLVQPGGSLRLSCAASGFTFSDSWIHWVRQAPGKGLEWVAWI
SPYGGSTYYADSVKGRFTISADTSKNTAYLQMNSLRAEDTAVYYCARRHWP
GGFDYWGQGTLVTVSS
or
(SEQ ID NO: 35)
EVQLVESGGGLVQPGGSLRLSCAASGFTFSDSWIHWVRQAPGKGLEWVAWI
SPYGGSTYYADSVKGRFTISADTSKNTAYLQMNSLRAEDTAVYYCARRHWP
GGFDYWGQGTLVTVSSASTK

and a light chain variable region comprising the amino acid sequence:

(SEQ ID NO: 36)
DIQMTQSPSSLSASVGDRVTITCRASQDVSTAVAWYQQKPGKAPKLLIYSA
SFLYSGVPSRFS GSGSGTDFTLTISSLQPEDFATYYCQQYLYHPATFGQG
TKVEIKR.

In some embodiments, PD-1 neutralizing agent comprises (i) a heavy chain or heavy chain variable region of SEQ ID NO: 37, or an amino acid sequence at least 50%, 60%, 70%, 80%, 90%, 95%, 98% or 99% identical thereto, and (ii) a light chain or light chain variable region of SEQ ID NO: 38, or an amino acid sequence at least 50%, 60%, 70%, 80%, 90%, 95%, 98% or 99% identical thereto.

(SEQ ID NO: 37)
EVQLVESGGGLVQPGGSLRLSCAASGFTFSDSWIHWVRQAPGKGLEWVAWI
SPYGGSTYYADSVKGRFTISADTSKNTAYLQMNSLRAEDTAVYYCARRHWP
GGFDYWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMIS
RTPEVTCVVVDVSHEDPEVKFNWYVDGVEVHNAKTKPREEQYASTYRVVSV
LTVLHQDWLNGKEYKCKVSNKALPAPIEKTISKAKGQPREPQVYTLPPSRE
EMTKQVSLTCLVKGFYPSDIAVEWESNGQPENYKTTPPVLDSDGSFFLYSK
LTVDKSRWQQGNVFSCSVMHEALHNHYTQKSLSLSPG
(SEQ ID NO: 38)
DIQMTQSPSSLSASVGDRVTITCRASQDVSTAVAWYQQKPGKAPKLLIYSA
SFLYSGVPSRFSGSGSGTDFTLTISSLQPEDFATYYCQQYLYHPATFGQGT
KVEIKRTVAAPSVFIEPPSDEQLKSGTASVVCLLNFYPREAKVQWKVDNAL
QSGNSQESVTEQDSKDSTYSLSSTLTLSKADYEKH KVYACEVTHQGLSSP
VTKSFNRGEC.

In some embodiments, the PD-1 neutralizing agent is an anti-PD-1 antibody that inhibits the binding of PD-1 to PD-L1. In some embodiments, the anti-PD-1 antibody is nivolumab. Nivolumab (also known as OPDIVO®; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., (2014) Cancer Immunol Res. 2(9):846-56). In another embodiment, the anti-PD-1 antibody or fragment thereof competes with nivolumab for binding to PD-1. In some embodiments, the anti-PD-1 antibody binds to the same epitope as nivolumab. In certain embodiments, the anti-PD-1 antibody has the same heavy and light chain CDRs as nivolumab.

In another embodiment, the anti-PD-1 antibody is pembrolizumab. Pembrolizumab (also known as “KEYTRUDA®”, lambrolizumab, and MK-3475) is a humanized monoclonal IgG4 antibody directed against human cell surface receptor PD-1. Pembrolizumab is described, for example, in U.S. Pat. No. 8,900,587. Pembrolizumab has been approved by the FDA for the treatment of relapsed or refractory melanoma and advanced NSCLC. In another embodiment, the anti-PD-1 antibody (or an antigen-binding portion thereof) competes with pembrolizumab for binding to PD-1. In some embodiments, the anti-PD-1 antibody binds to the same epitope as pembrolizumab. In certain embodiments, the anti-PD-1 antibody has the same heavy and light chain CDRs as pembrolizumab.

In some embodiments, the chemotherapy agent comprises at least one of a FOLFOX agent or a FOLFIRI agent, or at least one of an active ingredient comprised in a FOLFOX agent or a FOLFIRI agent. In some embodiments, the chemotherapy agent comprises a FOLFOX agent or a FOLFIRI agent. In some embodiments, the FOLFOX agent comprises oxaliplatin, 5-fluorouracil and leucovorin (also called “folinic acid”). In some embodiments, the FOLFIRI agent comprises irinotecan, 5-fluorouracil and leucovorin (also called “folinic acid”).

FOLFOX is a standard chemotherapy regimen for treatment of colorectal cancer. It comprises the following drugs: (i) folinic acid (leucovorin), a vitamin B derivative used as a “rescue” drug for high doses of the drug methotrexate but increases the cytotoxicity of 5-fluorouracil, (ii) fluorouracil (5-FU), a pyrimidine analog and antimetabolite which incorporates into the DNA molecule and stops synthesis, and (iii) oxaliplatin (Eloxatin).

FOLFOX4 is an adjuvant treatment in patients with stage III colon cancer, recommended for 12 cycles, every 2 weeks. The recommended dose schedule given every two weeks is as follows:
Day 1: Oxaliplatin 85 mg/m² intravenous infusion in 250-500 ml D5W (5% dextrose in water) and leucovorin 200 mg/m² intravenous infusion in D5W both given over 120 minutes at the same time in separate bags using a Y-line, followed by 5-FU 400 mg/m² intravenous bolus given over 2-4 minutes, followed by 5-FU 600 mg/m² intravenous infusion in 500 mL D5W (recommended) as a 22-hour continuous infusion.
Day 2: Leucovorin 200 mg/m² intravenous infusion over 120 minutes, followed by 5-FU 400 mg/m² intravenous bolus given over 2-4 minutes, followed by 5-FU 600 mg/m² intravenous infusion in 500 mL D5W (recommended) as a 22-hour continuous infusion.

FOLFOX6 is another standard chemotherapy regimen comprising folinic acid, fluorouracil (5-FU), and oxaliplatin, with the following standard regimen:

The dose schedule given every two weeks is as follows:
Day 1-2: Oxaliplatin 100 mg/m² IV infusion, given as a 120 minutes IV infusion in 500 mL D5W, concurrent with leucovorin 400 mg/m² (or levoleucovorin 200 mg/m²) IV infusion, followed by Fluorouracil 5-FU 400 mg/m² IV bolus, followed by 46-hour Fluorouracil 5-FU infusion (2400 mg/m² for first two cycles, increased to 3000 mg/m² in case of no toxicity > grade 1 during the first two cycles).
Days 3-14: Rest days

mFOLFOX6 is a modified FOLFOX regimen comprised of folinic acid, fluorouracil and oxaliplatin. The recommended mFOLFOX6 regimen is as follows, being understood that the skilled person may adapt this regimen according to local practice or the subject's case:
Oxaliplatin 85 mg/m² administered by IV infusion on Day 1
Folinic acid 400 mg/m² administered by IV infusion on Day 1
Fluorouracil 400 mg/m² administered by IV bolus on Day 1 followed by 2400 mg/m² administered by continuous IV infusion over 46 to 48 hours starting on Day 1.
   This regimen can be administered Q2W.

FOLFIRI is another chemotherapy regimen for treatment of colorectal cancer. It comprises the following drugs (Chen et al., (2016) Medicine. 95 (46): e5221): (i) folinic acid (leucovorin), a vitamin B derivative used as a “rescue” drug for high doses of the drug methotrexate but increases the cytotoxicity of 5-fluorouracil, (ii) fluorouracil (5-FU), a pyrimidine analog and antimetabolite which incorporates into the DNA molecule and stops synthesis, and (iii) irinotecan (Camptosar), a topoisomerase inhibitor, which prevents DNA for uncoiling and duplicating. The recommended FOLFIRI treatment regimen comprises:
Irinotecan 180 mg/m² administered by IV infusion on Day 1
Folinic acid 400 mg/m² administered by IV infusion on Day 1
Fluorouracil 400 mg/m² administered by IV bolus on Day 1 followed by 2400 mg/m² administered by continuous IV infusion over 46 to 48 hours starting on Day 1.
   This regimen can be administered Q2W.

“Reduces the inhibitory activity of VEGF”, “neutralizes VEGF” or “neutralizes the inhibitory activity of VEGF” refers to a process in which VEGF is inhibited.

In some embodiments, the VEGF neutralizing agent is an antibody or an antigen-binding fragment thereof that binds human vascular endothelial growth factor (VEGF).

In some embodiments, the VEGF neutralizing agent is bevacizumab. Bevacizumab (Avastin™) is a recombinant humanized monoclonal antibody directed against human vascular endothelial growth factor (VEGF). Disclosure related to bevacizumab can be found in U.S. Pat. Nos. 6,884,879, 7,060,269 and 7,297,334, which are incorporated herein by reference. Bevacizumab has the heavy and light chains of amino acid sequences SEQ ID NO: 39 and SEQ ID NO: 40, respectively.

In another embodiment, the VEGF neutralizing agent is an anti-VEGF antibody (or an antigen-binding portion thereof) competing with bevacizumab for binding to VEGF. In some embodiments, the anti-VEGF antibody binds to the same epitope as bevacizumab. In certain embodiments, the anti-VEGF antibody has the same heavy and light chain CDRs as bevacizumab.

In some embodiments, the VEGF neutralizing agent (e.g. an agent derived from bevacizumab) comprises (i) the heavy chain of SEQ ID NO: 39, or an amino acid sequence at least 50%, 60%, 70%, 80%, 90%, 95%, 98% or 99% identical thereto, and (ii) the light chain of SEQ ID NO: 40, or an amino acid sequence at least 50%, 60%, 70%, 80%, 90%, 95%, 98% or 99% identical thereto.

In some embodiments, the VEGF neutralizing agent comprises the heavy chain H-CDR1, H-CDR2 and H-CDR3 domains having the amino acid sequences of SEQ ID NOS:41-43, respectively, and the light chain L-CDR1, L-CDR2, L-CDR3 domains having the amino acid sequences of SEQ ID NOS: 44-46, respectively.

Bevacizumab heavy chain
(SEQ ID NO: 39)
EVQLVESGGGLVQPGGSLRLSCAASGYTFTNYGMNWVRQAPGKGLEWVGWI
NTYTGEPTYAADFKRRFTFSLDTSKSTAYLQMNSLRAEDTAVYYCAKYPHY
YGSSHWYFDVWGQGTLVTVSSASTKGPSVFPLAPSSKSTSGGTAALGCLVK
DYFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPGK
Bevacizumab light chain
(SEQ ID NO: 40)
DIQMTQSPSSLSASVGDRVTITCSASQDISNYLNWYQQKPGKAPKVLIYFT
SSLHSGVPSRFSGSGSGTDFTLTISSLQPEDFATYYCQQYSTVPWTFGQGT
KVEIKRTVAAPSVFIFPPSDEQLKSGTASVVCLLNNFYPREAKVQWKVDNA
LQSGNSQESVTEQDSKDSTYSLSSTLTLSKADYEKEIKVYACEVTHQGLSS
PVTKSFNRGEC
Bevacizumab, Heavy chain CDRs
H-CDR1:
(SEQ ID NO: 41)
GYTFTNYGMN
H-CDR2:
(SEQ ID NO: 42)
WINTYTGEPTYAADFKR
H-CDR3:
(SEQ ID NO: 43)
YPHYYGSSHWYFDV
Bevacizumab, Light Chain CDRs
L-CDR1:
(SEQ ID NO: 44)
SASQDISNYLN
L-CDR2:
(SEQ ID NO: 45)
FTSSLHS
L-CDR3:
(SEQ ID NO: 46)
QQYSTVPWT

It will be appreciated that, while the aforementioned antibodies can be used, other antibodies can recognize and be raised against any part of the VEGF polypeptide so long as the antibody causes the neutralization of the inhibitory activity of VEGF. For example, any fragment of VEGF, can be used as immunogens to raise antibodies, and the antibodies can recognize epitopes at any location within the VEGF polypeptide. In some embodiments, the epitope is the epitope specifically recognized by an antibody having a heavy chain variable CDRs of SEQ ID NOS: 41-43 and a light chain variable CDRs of SEQ ID NO: 44-46.

“Reduces the activity of EGFR”, “neutralizes EGFR” or “neutralizes the activity of EGFR” refers to a process in which EGFR is inhibited.

In some embodiments, the EGFR neutralizing agent is an antibody or an antigen-binding fragment thereof that binds human epidermal growth factor receptor (EGFR).

In some embodiments, the EGFR neutralizing agent is cetuximab. Cetuximab (Erbitux™) is a recombinant human/mouse chimeric monoclonal antibody directed against human epidermal growth factor receptor (EGFR, HER1, c-ErbB-1). Disclosure related to cetuximab can be found in U.S. Pat. No. 6,217,866 which is incorporated herein by reference. Cetuximab has the heavy and light chains of amino acid sequences SEQ ID NO: 47 and SEQ ID NO: 48, respectively.

In another embodiment, the EGFR neutralizing agent is an anti-EGFR antibody (or an antigen-binding portion thereof) competing with cetuximab for binding to EGFR. In some embodiments, the anti-EGFR antibody binds to the same epitope as cetuximab. In certain embodiments, the anti-EGFR antibody has the same heavy and light chain CDRs as cetuximab.

In some embodiments, the EGFR neutralizing agent (e.g. an agent derived from cetuximab) comprises (i) the heavy chain of SEQ ID NO: 47, or an amino acid sequence at least 50%, 60%, 70%, 80%, 90%, 95%, 98% or 99% identical thereto, and (ii) the light chain of SEQ ID NO: 48, or an amino acid sequence at least 50%, 60%, 70%, 80%, 90%, 95%, 98% or 99% identical thereto.

In some embodiments, the EGFR neutralizing agent comprises the heavy chain H-CDR1, H-CDR2 and H-CDR3 domains having the amino acid sequences of SEQ ID NOS:49-51, respectively, and the light chain L-CDR1, L-CDR2, L-CDR3 domains having the amino acid sequences of SEQ ID NOS: 52-54, respectively.

Cetuximab heavy chain
(SEQ ID NO: 47)
QVQLKQSGPGLVQPSQSLSITCTVSGFSLTNYGVHWVRQSPGKGLEWLGVI
WSGGNTDYNTPFTSRLSINKDNSKSQVFFKMNSLQSNDTAIYYCARALTYY
DYEFAYWGQGTLVTVSAASTKGPSVFPLAPSSKSTSGGTAALGCLVKDYFP
EPVTVSWNSGALTSGVHTFPAVLQSSGLYSLSSVVTVPSSSLGTQTYICNV
NHKPSNTKVDKRVEPKSCDKTHTCPPCPAPELLGGPSVFLFPPKPKDTLMI
SRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
Cetuximab light chain
(SEQ ID NO: 48)
DILLTQSPVILSVSPGERVSFSCRASQSIGTNIHWYQQRTNGSPRLLIKYA
SESISGIPSRFSGSGSGTDFTLSINSVESEDIADYYCQQNNNWPTTFGAGT
KLELKRTVAAPSVFIFPPSDEQLKSGTASVVCLLNNFYPREAKVQWKVDNA
LQSGNSQESVIEQDSKDSTYSLSSTLTLSKADYEKHKVYACEVTHQGLSSP
VTKSFNRGA
Cetuximab, Heavy chain CDRs
H-CDR1:
(SEQ ID NO: 49)
NYGVH
H-CDR2:
(SEQ ID NO: 50)
VIWSGGNTDYNTPFTS
H-CDR3:
(SEQ ID NO: 51)
ALTYYDYEFAY
Cetuximab, Light Chain CDRs
L-CDR1:
(SEQ ID NO: 52)
RASQSIGTNIH
L-CDR2:
(SEQ ID NO: 53)
YASESIS
L-CDR3:
(SEQ ID NO: 54)
QQNNNWPTT

It will be appreciated that, while the aforementioned antibodies can be used, other antibodies can recognize and be raised against any part of the EGFR polypeptide so long as the antibody causes the neutralization of the inhibitory activity of EGFR. For example, any fragment of EGFR, can be used as immunogens to raise antibodies, and the antibodies can recognize epitopes at any location within the EGFR polypeptide. In some embodiments, the epitope is the epitope specifically recognized by an antibody having a heavy chain variable CDRs of SEQ ID NOS: 49-51 and a light chain variable CDRs of SEQ ID NO: 52-54.

A “disorder” refers to any condition that would benefit from treatment using the methods of the disclosure. “Disorder” and “condition” are used interchangeably herein and include chronic and acute disorders or diseases, including those pathological conditions that predispose a patient to the disorder in question.

The term “subject” is intended to include human and non-human animals, particularly mammals. In certain embodiments, the subject is a human patient.

In some embodiments, the methods disclosed herein relate to treating a subject for a tumor disorder and/or a cancer disorder. In some embodiments, the cancer is colorectal cancer, colon cancer or rectal cancer

The terms “treatment” or “treat” as used herein refer to both therapeutic treatment and prophylactic or preventative measures. Those in need of treatment include subjects having cancer as well as those prone to having cancer or those in whom cancer is to be prevented. In some embodiments, the methods disclosed herein can be used to treat cancer. In other embodiments, those in need of treatment include subjects having a tumor as well as those prone to have a tumor or those in which a tumor is to be prevented. In certain embodiments, the methods disclosed herein can be used to treat tumors. In other embodiments, treatment of a tumor includes inhibiting tumor growth, promoting tumor reduction, or both inhibiting tumor growth and promoting tumor reduction.

The terms “administration” or “administering” as used herein refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

The terms “co-administered” or “in combination” as used herein refer to simultaneous or sequential administration of multiple compounds or agents. A first compound or agent may be administered before, concurrently with, or after administration of a second compound or agent. The first compound or agent and the second compound or agent may be simultaneously or sequentially administered on the same day, or may be sequentially administered within 1 day, 2 days, 3 days, 4 days, 5 days, 6 days, 1 week, 2 weeks, 3 weeks, or 1 month of each other. In some embodiments, compounds or agents are co-administered during the period in which each of the compounds or agents are exerting at least some physiological effect and/or has remaining efficacy.

Whenever “treatment of cancer” or the like is mentioned with reference to the agents disclosed herewith, are comprised:

(a) a method of treatment of cancer, said method comprising the step of administering (for at least one treatment) an NKG2A neutralizing agent, a PD-1 neutralizing agent, a VEGF neutralizing agent and a chemotherapy agent, (e.g., together or each separately in a pharmaceutically acceptable carrier material) to an individual, a mammal, especially a human, in need of such treatment, in a dose that allows for the treatment of cancer, (a therapeutically effective amount), optionally in a dose (amount) as specified herein;

(b) the use of a NKG2A neutralizing agent, a PD-1 neutralizing agent, a VEGF neutralizing agent and a chemotherapy agent, for the treatment of cancer;

(c) a NKG2A neutralizing agent, a PD-1 neutralizing agent, a VEGF neutralizing agent, and a chemotherapy agent, for use in the treatment of cancer (especially in a human);

(d) a NKG2A neutralizing agent for use in the treatment of cancer (especially in a human), wherein said NKG2A neutralizing agent is administered in combination with a PD-1 neutralizing agent, a VEGF neutralizing agent and a chemotherapy agent;

(e) a PD-1 neutralizing agent for use in the treatment of cancer (especially in a human), wherein said PD-1 neutralizing agent is administered in combination with a NKG2A neutralizing agent, a VEGF neutralizing agent and a chemotherapy agent;

(f) a VEGF neutralizing agent for use in the treatment of cancer (especially in a human), wherein said a VEGF neutralizing agent is administered in combination with a NKG2A neutralizing agent, a PD-1 neutralizing agent and a chemotherapy agent;

(g) a chemotherapy agent for use in the treatment of cancer (especially in a human), wherein said chemotherapy agent is administered in combination with a NKG2A neutralizing agent, a PD-1 neutralizing agent and a VEGF neutralizing agent;

(h) the use of a NKG2A neutralizing agent, a PD-1 neutralizing agent, a VEGF neutralizing agent and a chemotherapy agent for the manufacture of a pharmaceutical preparation for the treatment of cancer,

(i) a method of using a NKG2A neutralizing agent, a PD-1 neutralizing agent, a VEGF neutralizing agent and/or a chemotherapy agent for the manufacture of a pharmaceutical preparation for the treatment of cancer, comprising admixing at least one of: a NKG2A neutralizing agent, a PD-1 neutralizing agent, a VEGF neutralizing agent, and a chemotherapy agent, with a pharmaceutically acceptable carrier,

(j) a pharmaceutical preparation comprising an effective dose of a NKG2A neutralizing agent and/or of a PD-1 neutralizing agent and/or a VEGF neutralizing agent and/or a chemotherapy agent that is appropriate for the treatment of cancer;

(k) any combination of (a) to (j), in accordance with the subject matter allowable for patenting in a country where this application is filed.

In any of (a) to (k) above, the reference to a VEGF neutralizing agent may be replaced with reference to an EGFR neutralizing agent. Accordingly whenever “treatment of cancer” or the like is mentioned with reference to the agents disclosed herewith, are comprised:

(l) a method of treatment of cancer, said method comprising the step of administering (for at least one treatment) an NKG2A neutralizing agent, a PD-1 neutralizing agent, an EGFR neutralizing agent and a chemotherapy agent, (e.g., together or each separately in a pharmaceutically acceptable carrier material) to an individual, a mammal, especially a human, in need of such treatment, in a dose that allows for the treatment of cancer, (a therapeutically effective amount), optionally in a dose (amount) as specified herein;

(m) the use of a NKG2A neutralizing agent, a PD-1 neutralizing agent, an EGFR neutralizing agent and a chemotherapy agent, for the treatment of cancer;

(n) a NKG2A neutralizing agent, a PD-1 neutralizing agent, an EFGR neutralizing agent, and a chemotherapy agent, for use in the treatment of cancer (especially in a human);

(o) a NKG2A neutralizing agent for use in the treatment of cancer (especially in a human), wherein said NKG2A neutralizing agent is administered in combination with a PD-1 neutralizing agent, an EGFR neutralizing agent and a chemotherapy agent;

(p) a PD-1 neutralizing agent for use in the treatment of cancer (especially in a human), wherein said PD-1 neutralizing agent is administered in combination with a NKG2A neutralizing agent, an EGFR neutralizing agent and a chemotherapy agent;

(q) an EGFR neutralizing agent for use in the treatment of cancer (especially in a human), wherein said EGFR neutralizing agent is administered in combination with a NKG2A neutralizing agent, a PD-1 neutralizing agent and a chemotherapy agent;

(r) a chemotherapy agent for use in the treatment of cancer (especially in a human), wherein said chemotherapy agent is administered in combination with a NKG2A neutralizing agent, a PD-1 neutralizing agent and an EGFR neutralizing agent;

(s) the use of a NKG2A neutralizing agent, a PD-1 neutralizing agent, an EGFR neutralizing agent and a chemotherapy agent for the manufacture of a pharmaceutical preparation for the treatment of cancer,

(t) a method of using a NKG2A neutralizing agent, a PD-1 neutralizing agent, an EGFR neutralizing agent and/or a chemotherapy agent for the manufacture of a pharmaceutical preparation for the treatment of cancer, comprising admixing at least one of: a NKG2A neutralizing agent, a PD-1 neutralizing agent, an EGFR neutralizing agent, and a chemotherapy agent, with a pharmaceutically acceptable carrier,

(u) a pharmaceutical preparation comprising an effective dose of a NKG2A neutralizing agent and/or of a PD-1 neutralizing agent and/or an EGFR neutralizing agent and/or a chemotherapy agent that is appropriate for the treatment of cancer;

(v) any combination of (1) to (u), in accordance with the subject matter allowable for patenting in a country where this application is filed.

Disclosed herein are methods useful in the diagnosis, prognosis, monitoring and treatment of a cancer, particularly colorectal cancer, for example advanced recurrent or metastatic colorectal cancer. In some embodiments, the cancer is characterized by tumors that are not DNA mismatch repair defective and/or that are microsatellite stable. Colorectal cancer (CRC) as used herein refers to colon cancer, rectal cancer, and colorectal cancer (cancer of both the colon and rectal areas).

Microsatellites are repeated sequences of DNA distributed throughout the genome. Although the length of these microsatellites is highly variable from person to person, each subject has microsatellites of a set length. These repeated sequences are common, and normal. The most common microsatellite in humans is a dinucleotide repeat of CA, which occurs tens of thousands of times across the genome. In cells with mutations in DNA repair genes, however, some of these sequences accumulate errors and become longer or shorter. The appearance of abnormally long or short microsatellites in a subject's DNA is referred to as microsatellite instability (MSI). Microsatellite instability is the condition of genetic hypermutability that results from impaired DNA mismatch repair (MMR). The presence of microsatellite instability (MSI) represents phenotypic evidence that MMR is not functioning normally. The absence of microsatellite instability is termed microsatellite stability (MSS).

MSI is a key factor in several cancers including colorectal, endometrial, ovarian and gastric cancers (Soreide et al. (2006) The British Journal of Surgery 93:395-406; Ali-Fehmi et al. (2006) International Journal of Gynecological Pathology 25:223-229; Vauhkonen et al. (2006) Clinical Gastroenterology 20:651-674).

Colorectal cancer studies have demonstrated two mechanisms for MSI occurrence. The first is in hereditary nonpolyposis colorectal cancer (HNPCC) or Lynch Syndrome, where an inherited mutation in a DNA mismatch-repair gene causes a microsatellite repeat replication error to go unfixed. The replication error results in a frameshift mutation that inactivates or alters major tumor suppressor genes and, ultimately, the prevention of cancer. The second mechanism whereby MSI causes colorectal cancer is an epigenetic change which silences an essential DNA mismatch-repair gene. In both cases, microsatellite insertions and deletions within tumor suppressor gene coding regions result in uncontrolled cell division and tumor growth.

Five markers have been recommended by the National Cancer Institute to screen for MSI in HNPCC tumors (often called “Bethesda markers”). These five markers of MSI presence are: two mononucleotide repeats BAT25 and BAT26, and three dinucleotide repeats D5S346, D2S123, and D17S250 (Umar et al (2004) Journal of the National Cancer Institute 96:261-268). Generally, MSI detection in two of the five “Bethesda markers” is considered a positive result or high probability of MSI (MSI-High or MSI-H). Standard methods for detecting MSI in biological samples include the use of Promega™'s microsatellite instability assay (MSI Analysis System) that includes five mononucleotide markers chosen for their sensitivity and specificity, these five markers are: BAT-25, BAT-26, NR-21, NR-24 and MON027 (Bacher et al. (2004) Disease Markers 20:237-250).

In most cases, the genetic basis for instability in MSI tumors is an inherited germline alteration in any one or more of the five human MMR genes: MSH2, MLH1, MSH6, PMS2, and PMS1.

Another MSI, called elevated microsatellite alterations at selected tetranucleotide repeats (EMAST), was recently discovered. However, EMAST is unique in that it is not derived from MMR, and it is commonly associated with TP53 mutations (Boland et al. (2010) Gastroenterology 138 (6): 2073-2087).

Thus, microsatellite instability in a tumor can be determined by assessing microsatellite markers and/or MMR genes.

In some embodiments, the subject has a tumor that is not microsatellite Instability-High (MSI-H) and/or not DNA mismatch repair (MMR) defective. In some embodiments, the subject has a tumor that does not have microsatellite instability detected in two or more microsatellite markers, wherein the subject has a tumor that has no alteration detected in two or more of the microsatellite markers selected from the group consisting of BAT-25, BAT-26, NR-21, NR-24, and MON027. In some embodiments, the subject has a tumor that does not have an alteration in expression of a DNA mismatch repair (MMR) protein, wherein the subject has a tumor that does not have decreased or absence of expression of at least one MMR protein selected from MSH2, MLH1, MSH6 and PMS2. In some embodiments, the subject has a tumor that is microsatellite stable (MSS). In some embodiments, the subject has a microsatellite stable-colorectal cancer (MSS-CRC).

The DNA mismatch repair status of a tumor, optionally the MMR status and/or microsatellite status in a subject can be measured prior to administering any composition or utilizing any method disclosed herein.

The agents and methods described herein may be used with or without a prior step of determining the DNA mismatch repair status of the tumor's subject, optionally by determining the MMR status and/or microsatellite status on cells in a biological sample obtained from the subject (e.g. a biological sample comprising cancer cells, cancer tissue or cancer-adjacent tissue).

A biological sample from a subject, for example from a biopsy, can be obtained and assessed. MMR status and/or microsatellite status can be determined by any methods known in the art, see, e.g., Umar et al. Journal of the National Cancer Institute 2004; 96(4):261-268 and Bacher et al. Disease Markers 2004; 20:237-250. In one embodiment, MMR status is assessed by immunohistochemical analysis demonstrating the presence or absence of expression of any one or more of the following proteins: MLH1, MSH2, MSH6, or PMS2. In one embodiment, microsatellite status is assessed by detecting high-frequency microsatellite instability in microsatellite markers, for example BAT-25, BAT-26, NR-21, NR-24, MON027, D5S346, D2S123, and D17S250. In one embodiment, microsatellite instability detected for two or more microsatellite markers, for example for BAT-25, BAT-26, NR-21, NR-24, and/or MON027, indicates a MSI-H status, while microsatellite instability for a single MSI marker or no instability for any of the MSI markers tested is interpreted as microsatellite instability-Low (MSI-L) and microsatellite stable (MSS), respectively.

In one embodiment, a tumor that is not DNA mismatch repair defective or that is MSS has no microsatellite instability or microsatellite instability detected at less than two or more microsatellite markers, for example BAT-25, BAT-26, NR-21, NR-24, or MON027, and no absence of protein expression at any one or more of proteins MLH1, MSH2, MSH6, or PMS2.

In one embodiment, MSI-H tumors have greater than at least about 30% of unstable MSI markers. In one embodiment, MSI-L tumors do have unstable MSI markers but less than about 10%, less than about 20%, or less than about 30% of the MSI markers of said tumors are unstable MSI markers. In one embodiment, MSS tumors have no unstable MSI marker. In some embodiments, a colorectal cancer is MSI-L when less than about 30%, less than about 20% or less than about 10% of the tested MSI markers exhibit instability. In some embodiments, a colorectal cancer is MSS when none of the tested MSI markers exhibit instability.

Mutations in the RAS gene family, particularly the KRAS isoform, have been reported as predictors of poor overall and recurrence-free survival in patients with CRC. The treatments of the invention may be used in subjects regardless of their RAS status (e.g. regardless of whether a subject is RAS wild-type or has at least one mutation in one or more RAS genes, such as the KRAS isoform). Alternatively, treatments of the invention may be used in subjects known to be wild-type for RAS, or in subjects known to have at least one mutation in one or more RAS genes, such as the KRAS isoform.

In some embodiments, a subject has a cancer that is resistant, has not responded, has relapsed and/or progressed despite (e.g. during or following) surgery and/or treatment with a therapeutic agent, e.g. a chemotherapeutic agent or radiotherapy.

The terms “first line treatment”, “1L treatment”, “1-L treatment”, “1L” or “1-L” as used herein refer to the first treatment given for a disease, particularly a cancer as described herein. A first line treatment may be specific for a given type or subtype of cancer, or a specific cancer stage. A first line treatment may be part of a standard set of treatments. A first-line treatment is generally accepted as the best treatment for a disease, particularly a cancer as described herein. If a first line treatment does not cure the disease or it causes severe side effects, subsequent lines of treatment may be used instead. In some embodiments, the disclosure relates to providing first line treatments for cancer (methods of treatment, or pharmaceutical formulations for use as described herein).

In some embodiments, the NKG2A neutralizing agent, the PD-1 neutralizing agent, the chemotherapy agent and the VEGF neutralizing agent or the EGFR neutralizing agent are administered simultaneously, separately, or sequentially. In some embodiments, the NKG2A neutralizing agent, the PD-1 neutralizing agent, the chemotherapy agent and the VEGF neutralizing agent or the EGFR neutralizing agent are formulated for separate administration and are administered concurrently or sequentially.

In one embodiment, provided is an agent that neutralizes NKG2A (optionally an anti-NKG2A antibody such as monalizumab), for use in the treatment of cancer (optionally colorectal cancer, e.g. mCRC), wherein the agent that neutralizes NKG2A is administered in combination with an agent that neutralizes PD-1 (optionally an anti-PD-1 or anti-PD-L1 antibody such as durvalumab), an agent that neutralizes VEGF (optionally an anti-VEGF antibody such as bevacizumab), and a chemotherapy agent (such as FOLFOX or FOLFIRI).

In one embodiment, provided is an agent that neutralizes a human PD-1 polypeptide (optionally an anti-PD-L1 antibody or an anti-PD-1 antibody such as durvalumab), for use in the treatment of cancer (optionally colorectal cancer, e.g. mCRC), wherein the agent that neutralizes a human PD-1 polypeptide is administered in combination with an agent that neutralizes NKG2A (optionally an anti-NKG2A antibody such as monalizumab), an agent that neutralizes VEGF (optionally an anti-VEGF antibody such as bevacizumab), and a chemotherapy agent (such as FOLFOX or FOLFIRI).

In one embodiment, provided is an agent that neutralizes VEGF (optionally an anti-VEGF antibody such as bevacizumab), for use in the treatment of cancer (optionally colorectal cancer, e.g. mCRC), wherein the agent that neutralizes VEGF is administered in combination with an agent that neutralizes NKG2A (optionally an anti-NKG2A antibody such as monalizumab), an agent that neutralizes a human PD-1 polypeptide (optionally an anti-PD-L1 antibody or an anti-PD-1 antibody such as durvalumab), and a chemotherapy agent (such as FOLFOX or FOLFIRI).

In one embodiment, provided is a chemotherapy agent (such as FOLFOX or FOLFIRI), for use in the treatment of cancer (optionally colorectal cancer, e.g. mCRC), wherein the chemotherapy agent is administered in combination with an agent that neutralizes NKG2A (optionally an anti-NKG2A antibody such as monalizumab), an agent that neutralizes a human PD-1 polypeptide (optionally an anti-PD-L1 antibody or an anti-PD-1 antibody such as durvalumab), and an agent that neutralizes VEGF (optionally an anti-VEGF antibody such as bevacizumab).

In a still other embodiment, provided is a pharmaceutical formulation comprising a therapeutically effective amount of a NKG2A neutralizing agent, a PD-1 neutralizing agent, a chemotherapy agent, and a VEGF neutralizing agent, for use in treating a subject who has a cancer (optionally a colorectal cancer), wherein the subject has a tumor that is not MSI-H and/or not DNA mismatch-repair (MMR) defective.

In a further embodiment, provided is a pharmaceutical formulation comprising a therapeutically effective amount of a NKG2A neutralizing agent, for use in treating a subject who has a cancer (optionally a colorectal cancer), wherein the subject has a tumor that is not MSI-H and/or not DNA mismatch-repair (MMR) defective, and wherein said pharmaceutical formulation is administered in combination with a PD-1 neutralizing agent, a chemotherapy agent, and a VEGF neutralizing agent.

In a further embodiment, provided is a pharmaceutical formulation comprising a therapeutically effective amount of a PD-1 neutralizing agent, for use in treating a subject who has a cancer (optionally a colorectal cancer), wherein the subject has a tumor that is not MSI-H and/or not DNA mismatch-repair (MMR) defective, and wherein said pharmaceutical formulation is administered in combination with a NKG2A neutralizing agent, a chemotherapy agent, and a VEGF neutralizing agent.

In a further embodiment, provided is a pharmaceutical formulation comprising a therapeutically effective amount of a chemotherapy agent, for use in treating a subject who has a cancer (optionally a colorectal cancer), wherein the subject has a tumor that is not MSI-H and/or not DNA mismatch-repair (MMR) defective, and wherein said pharmaceutical formulation is administered in combination with a NKG2A neutralizing agent, a PD-1 neutralizing agent, and a VEGF neutralizing agent.

In a further embodiment, provided is a pharmaceutical formulation comprising a therapeutically effective amount of a VEGF neutralizing agent, for use in treating a subject who has a cancer (optionally a colorectal cancer), wherein the subject has a tumor that is not MSI-H and/or not DNA mismatch-repair (MMR) defective, and wherein said pharmaceutical formulation is administered in combination with a NKG2A neutralizing agent, a PD-1 neutralizing agent, and a chemotherapy agent.

In one embodiment, provided is an agent that neutralizes NKG2A (optionally an anti-NKG2A antibody such as monalizumab), for use in the treatment of cancer (optionally colorectal cancer, e.g. mCRC), wherein the agent that neutralizes NKG2A is administered in combination with an agent that neutralizes PD-1 (optionally an anti-PD-1 or anti-PD-L1 antibody such as durvalumab), an agent that neutralizes EGFR (optionally an anti-EGFR antibody such as cetuximab), and a chemotherapy agent (such as FOLFOX or FOLFIRI).

In one embodiment, provided is an agent that neutralizes a human PD-1 polypeptide (optionally an anti-PD-L1 antibody or an anti-PD-1 antibody such as durvalumab), for use in the treatment of cancer (optionally colorectal cancer, e.g. mCRC), wherein the agent that neutralizes a human PD-1 polypeptide is administered in combination with an agent that neutralizes NKG2A (optionally an anti-NKG2A antibody such as monalizumab), an agent that neutralizes EGFR (optionally an anti-EGFR antibody such as cetuximab), and a chemotherapy agent (such as FOLFOX or FOLFIRI).

In one embodiment, provided is an agent that neutralizes EGFR (optionally an anti-EGFR antibody such as cetuximab), for use in the treatment of cancer (optionally colorectal cancer, e.g. mCRC), wherein the agent that neutralizes EGFR is administered in combination with an agent that neutralizes NKG2A (optionally an anti-NKG2A antibody such as monalizumab), an agent that neutralizes a human PD-1 polypeptide (optionally an anti-PD-L1 antibody or an anti-PD-1 antibody such as durvalumab), and a chemotherapy agent (such as FOLFOX or FOLFIRI).

In one embodiment, provided is a chemotherapy agent (such as FOLFOX or FOLFIRI), for use in the treatment of cancer (optionally colorectal cancer, e.g. mCRC), wherein the chemotherapy agent is administered in combination with an agent that neutralizes NKG2A (optionally an anti-NKG2A antibody such as monalizumab), an agent that neutralizes a human PD-1 polypeptide (optionally an anti-PD-L1 antibody or an anti-PD-1 antibody such as durvalumab), and an agent that neutralizes EGFR (optionally an anti-EGFR antibody such as cetuximab).

In a still other embodiment, provided is a pharmaceutical formulation comprising a therapeutically effective amount of a NKG2A neutralizing agent, a PD-1 neutralizing agent, a chemotherapy agent, and an EGFR neutralizing agent, for use in treating a subject who has a cancer (optionally a colorectal cancer), wherein the subject has a tumor that is not MSI-H and/or not DNA mismatch-repair (MMR) defective.

In a further embodiment, provided is a pharmaceutical formulation comprising a therapeutically effective amount of a NKG2A neutralizing agent, for use in treating a subject who has a cancer (optionally a colorectal cancer), wherein the subject has a tumor that is not MSI-H and/or not DNA mismatch-repair (MMR) defective, and wherein said pharmaceutical formulation is administered in combination with a PD-1 neutralizing agent, a chemotherapy agent, and an EGFR neutralizing agent.

In a further embodiment, provided is a pharmaceutical formulation comprising a therapeutically effective amount of a PD-1 neutralizing agent, for use in treating a subject who has a cancer (optionally a colorectal cancer), wherein the subject has a tumor that is not MSI-H and/or not DNA mismatch-repair (MMR) defective, and wherein said pharmaceutical formulation is administered in combination with a NKG2A neutralizing agent, a chemotherapy agent, and an EGFR neutralizing agent.

In a further embodiment, provided is a pharmaceutical formulation comprising a therapeutically effective amount of a chemotherapy agent, for use in treating a subject who has a cancer (optionally a colorectal cancer), wherein the subject has a tumor that is not MSI-H and/or not DNA mismatch-repair (MMR) defective, and wherein said pharmaceutical formulation is administered in combination with a NKG2A neutralizing agent, a PD-1 neutralizing agent, and an EGFR neutralizing agent.

In a further embodiment, provided is a pharmaceutical formulation comprising a therapeutically effective amount of an EGFR neutralizing agent, for use in treating a subject who has a cancer (optionally a colorectal cancer), wherein the subject has a tumor that is not MSI-H and/or not DNA mismatch-repair (MMR) defective, and wherein said pharmaceutical formulation is administered in combination with a NKG2A neutralizing agent, a PD-1 neutralizing agent, and a chemotherapy agent.

The terms “pharmaceutical composition” or “therapeutic composition” as used herein refer to a compound or composition capable of inducing a desired therapeutic effect when properly administered to a subject. In some embodiments, the disclosure provides a pharmaceutical composition comprising a pharmaceutically acceptable carrier and a therapeutically effective amount of at least one inhibitor of the disclosure.

The terms “pharmaceutically acceptable carrier” or “physiologically acceptable carrier” as used herein refer to one or more formulation materials suitable for accomplishing or enhancing the delivery of one or more agents of the disclosure.

In some embodiments, the agents disclosed herein may be formulated with a pharmaceutically acceptable carrier, excipient, or stabilizer, as pharmaceutical compositions. In certain embodiments, such pharmaceutical compositions are suitable for administration to a human or non-human animal via any one or more routes of administration using methods known in the art. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances which are suitable for administration into a human. Other contemplated carriers, excipients, and/or additives, which may be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium, and the like. These and additional known pharmaceutical carriers, excipients, and/or additives suitable for use in the formulations described herein are known in the art, e.g., as listed in “Remington: The Science & Practice of Pharmacy,” 21st ed., Lippincott Williams & Wilkins, (2005), and in the “Physician's Desk Reference,” 60th ed., Medical Economics, Montvale, N.J. (2005). Pharmaceutically acceptable carriers can be selected that are suitable for the mode of administration, solubility, and/or stability desired or required.

In one embodiment, the formulations of the disclosure are pyrogen-free formulations that are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension, and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one-hour period for intravenous drug applications (The United States Pharmacopeial Convention, Pharmacopeial Forum 26(1): 223 (2000)). In certain embodiments, the endotoxin and pyrogen levels in the composition are less than 10 EU/mg, or less than 5 EU/mg, or less than 1 EU/mg, or less than 0.1 EU/mg, or less than 0.01 EU/mg, or less than 0.001 EU/mg.

When used for in vivo administration, the formulations of the disclosure should be sterile. The formulations of the disclosure may be sterilized by various sterilization methods, including, for example, sterile filtration or radiation. In one embodiment, the formulation is filter sterilized with a presterilized 0.22-micron filter. Sterile compositions for injection can be formulated according to conventional pharmaceutical practice as described in “Remington: The Science & Practice of Pharmacy,” 21st ed., Lippincott Williams & Wilkins, (2005).

In some embodiments, therapeutic compositions can be formulated for particular routes of administration, such as oral, nasal, pulmonary, topical (including buccal and sublingual), rectal, vaginal, and/or parenteral administration. The terms “parenteral administration” and “administered parenterally” as used herein refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion. Formulations of the disclosure that are suitable for topical or transdermal administration include powders, sprays, ointments, pastes, creams, lotions, gels, solutions, patches, and inhalants. The inhibitors and other actives may be mixed under sterile conditions with a pharmaceutically acceptable carrier, and with any preservatives, buffers, or propellants which may be required (see, e.g., U.S. Pat. Nos. 7,378,110; 7,258,873; and 7,135,180; U.S. Patent Application Publication Nos. 2004/0042972 and 2004/0042971).

The formulations can be presented in unit dosage form and can be prepared by any method known in the art of pharmacy. Actual dosage levels of the active ingredients in the pharmaceutical compositions of the present disclosure may be varied so as to obtain an amount of the active ingredient which is effective to achieve the desired therapeutic response for a particular patient, composition, and mode of administration, without being toxic to the patient (e.g., “a therapeutically effective amount”). The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. These dosages may be administered daily, weekly, biweekly, monthly, or less frequently, for example, biannually, depending on dosage, method of administration, disorder or symptom(s) to be treated, and subject characteristics. Dosages can also be administered via continuous infusion (such as through a pump). The administered dose may also depend on the route of administration. For example, subcutaneous administration may require a higher dosage than intravenous administration. As noted above, any commonly used dosing regimen (e.g., 1-10 mg/kg administered by injection or infusion daily or twice a week) may be adapted and suitable in the methods relating to treating human cancer patients.

The combination therapy dose of NKG2A neutralizing agent, a PD-1 neutralizing agent, a chemotherapy agent, and a VEGF neutralizing agent or EGFR neutralizing agent will vary depending, in part, upon the size (body weight, body surface, or organ size) and condition (the age and general health) of the patient.

In some embodiments, NKG2A neutralizing agent is monalizumab, the PD-1 neutralizing agent is durvalumab, the chemotherapy agent comprises folinic acid, fluorouracil, and oxaliplatin, and the VEGF neutralizing agent is bevacizumab. In some embodiments, the NKG2A neutralizing agent is monalizumab administered at a fixed dose of 750 mg every 2 weeks, the PD-1 neutralizing agent is durvalumab administered at a fixed dose of 1500 mg/kg every 4 weeks, the chemotherapy agent comprises folinic acid administered at a fixed dose of 400 mg/m², fluorouracil administered at a fixed dose of 400 mg/m² bolus followed by 2400 mg/m² continuous IV infusion, and oxaliplatin administered at a fixed dose of 85 mg/m² every 2 weeks, and the VEGF neutralizing agent is bevacizumab administered at a fixed dose of 5 mg/kg every 2 weeks.

In some embodiments, NKG2A neutralizing agent is monalizumab, the PD-1 neutralizing agent is durvalumab, the chemotherapy agent comprises folinic acid, fluorouracil, and oxaliplatin, and the EGFR neutralizing agent is cetuximab. In some embodiments, the NKG2A neutralizing agent is monalizumab administered at a fixed dose of 750 mg every 2 weeks, the PD-1 neutralizing agent is durvalumab administered at a fixed dose of 1500 mg/kg every 4 weeks, the chemotherapy agent comprises folinic acid administered at a fixed dose of 400 mg/m², fluorouracil administered at a fixed dose of 400 mg/m² bolus followed by 2400 mg/m² continuous IV infusion, and oxaliplatin administered at a fixed dose of 85 mg/m² every 2 weeks, and the EGFR neutralizing agent is cetuximab administered as a fixed dose of 500 mg/m² every two weeks.

Without limiting the disclosure, a number of embodiments of the disclosure are described herein for purpose of illustration.

### EXAMPLES

The Examples that follow are illustrative of specific embodiments of the disclosure, and various uses thereof. They are set forth for explanatory purposes only and should not be construed as limiting the scope of the disclosure in any way.

### Example 1. Combination of Chemotherapeutic Regimens and Bevacizumab for Treatment of CRC

Bevacizumab, a humanized monoclonal antibody that blocks the activity of vascular endothelial growth factor (VEGF), a factor that plays an important role in tumor angiogenesis, was first approved as a treatment for mCRC in 2004. It is well suited for use in combination with first- or second-line chemotherapy in the treatment of mCRC because its side effects are predictable and appear not to add to the incidence or severity of the side effects of chemotherapy (Hochster et al (2008) J. Clin. Oncol. 26:3523-3529). Clinical trials of bevacizumab in combination with oxaliplatin-containing and 5-fluorouracil-based regimens have shown that combination therapy is well tolerated, and its toxicity is not substantially greater than that of the chemotherapy alone (Ducreux et al (2013) Eur J Cancer 49:1236-1245; Tol et al (2008) Ann Oncol. 19:734-8). Some randomized trials showed that bevacizumab improved response rates, overall survival, and progression-free survival in colorectal cancer metastatic patients when combined with the different standard chemotherapy regimens (Prescrire Int. 2006; 15:94-7; Hurwitz et al (2004) N Engl J Med.350:2335-42). Results have also been reported from a large, head-to-head, randomized, double-blind, placebo-controlled, phase III study (NO16966) in which CapeOx (capecitabine dose, 1000 mg/m², twice daily for 14 days) with bevacizumab or placebo was compared with FOLFOX with bevacizumab or placebo in patients with unresectable metastatic disease (Saltz et al (2008) J Clin Oncol. 26:2013-2019). The addition of bevacizumab to oxaliplatin-based regimens was associated with a more modest increase of 1.4 months in Progression-free survival (PFS) compared with regimens without bevacizumab and the difference in Overall Survival (OS), which was also a modest 1.4 months, which did not reach statistical significance.

### Example 2. PD-1 Neutralizing Agents for Treatment of CRC

i. PD-1 Neutralizing Agents Alone in CRC

Monotherapy with checkpoint inhibitors in subjects with MSS-CRC has resulted in limited or no antitumor activity. For example, an objective response rate (ORR) of 0% was reported when pembrolizumab 10 mg/kg Q2W was administered to subjects with microsatellite proficient CRC (Le D T et al (2015) N Engl J Med. 372(26):2509-20). On the other hand, studies of monotherapy with pembrolizumab 200 mg Q3W or 10 mg/kg Q2W and nivolumab 3 mg/kg Q2W in subjects with microsatellite instability-high CRC or mismatch repair deficient CRC have reported an ORR of 36% for pembrolizumab (FDA, 2017a, US Food and Drug Administration. https://www.fda.gov/Drugs/InformationOnDrugs/ApprovedDrugs/ucm560040.htm) and 28% for nivolumab (FDA, 2017b, US Food and Drug Administration. https://www.fda.gov/Drugs/InformationOnDrugs/ApprovedDrugs/ucm569366.htm). In patients with microsatellite instability-high (MSI-H) metastatic colorectal cancer, the inhibition of programmed death-1 (PD-1) pathway has achieved promising responses (Rosenbaum et al (2016) Mod Pathol 29: 1104-1112). PD-1 is an immune inhibitory receptor, expressed in many cells, including T cells. Its ligand, PD-L1, is expressed on surface of several cell types, especially tumor cells. When PD-L1 binds to PD-1, an inhibitory signal is transmitted into the T cell, which suppresses T-cell proliferation. MSI-H metastatic CRC gives rise to high percentage of mutations which is proportional to mutational load. High mutational load of MSI-H CRC correlates with increased PD-L1 expression which indicates a higher likelihood of response to PD-1 inhibitors, compared to microsatellite instability-stable (MSI-S) CRC (Boland et al (2010) Gastroenterology 138: 2073-2087; Champiat et al (2014) Oncoimmunology 3:e27817; Le D T et al (2015) N Engl J Med 372: 2509-2520). Thus, MSI-H CRC could respond to single agent PD-1 pathway inhibition.

ii. PD-1 Neutralizing Agents in Combination with Chemotherapy in CRC

Anti-PD-1 agents in combination with chemotherapy have been tested in subjects with metastatic CRC. A Phase 1b study evaluated nivolumab (3 mg/kg on days land 15 every 28-day cycle) in combination with capecitabine (1000 mg orally twice daily days 1 to 5 on, days 6 to 7 off, each 7-day period) and irinotecan (175 mg/m2 on day 1 every 14 days) in subjects with previously treated, metastatic CRC. Subjects were treated until disease progression or toxicity. All of the 9 subjects for whom data were available had treatment-related adverse events (any grade). The most common (>50% subjects) were fatigue (Grade 1), nausea (Grade 1), and diarrhea (Grade 1). No IRRs were observed. There were no dose limiting toxicities or study-related serious adverse events. Of 6 subjects evaluable for best overall response, 1 had a PR for 8 months, 1 had SD for 6 months, 1 had ongoing SD over 3 months, and 3 had disease progression. It was concluded that nivolumab in combination with capecitabine and irinotecan appeared to be safe in subjects with previously treated, metastatic CRC (Khemka et al (2016) Ann Oncol. 27(Suppl 2):ii80-1).

The mFOLFOX6 chemotherapy regimen in combination with pembrolizumab was evaluated in a Phase 2 study that enrolled subjects with untreated, unresectable CRC. During the safety run-in, 2 patients had Grade 3 febrile neutropenia and 1 had Grade 4 neutropenia. Consequently, the data safety monitoring committee recommended 20% dose reduction of the mFOLFOX6 regimen. Of the 27 evaluable subjects, 36.7% experienced Grade 3-4 toxicity during the median follow-up period of 24 weeks. Febrile neutropenia was not reported during the follow-up period and there was no Grade 5 toxicity observed. Reported responses were 1 CR, 15 PRs, (ORR=53%) and 14 SD. The authors concluded that combination therapy with mFOLFOX6 and pembrolizumab had acceptable toxicity in subjects with untreated, advanced CRC and clinical activity was demonstrated (Shanda et al (2017) J Clin Oncol. 35(15-Suppl):3541)

iii. PD-1 Neutralizing Agents in Combination with Chemotherapy and Bevacizumab in CRC

In an open-label Phase 1b study, subjects with refractory, metastatic CRC were treated with atezolizumab (an anti-PD-1 agent) 20 mg/kg Q3W in combination with bevacizumab 15 mg/kg Q3W (Arm A) and a group of oxaliplatin-naïve subjects with metastatic CRC received atezolizumab 14 mg/kg Q2W in combination with bevacizumab 10 mg/kg Q2W and folinic acid, fluorouracil, and oxaliplatin (FOLFOX) at standard doses (Arm B). In Arm A (n=14), Grade 3 to 4 AEs regardless of attribution were 64%, including abdominal pain, hyperbilirubinemia and pneumonia (14% each). In Arm B (n=30), 73% of subjects had Grade 3 to 4 AEs, including neutropenia (40%), diarrhea (13%), increased ALT (10%) and increased AST (10%). Grade ≥3 atezolizumab-related AEs were 7% in Arm A and 20% in Arm B. For subjects with ≥1 tumor assessment, the unconfirmed ORR was 8% (1/13) in Arm A and 36% (9/25) in Arm B. The unconfirmed ORR was 44% (8/18) for Arm B first-line (1L) subjects. Minimum follow-up was 1.9 months in Arm A and 2.2 months in Arm B.

It was concluded that atezolizumab in combination with bevacizumab with or without FOLFOX was well tolerated with no unexpected toxicities. Clinical activity was observed with both treatment combinations (Bendell et al (2015) J Clin Oncol. 33(Suppl 3); Abstract 704).

### Example 3. Combination of a PD-1 Neutralizing Agent and a NKG2A Neutralizing Agent for Treatment of CRC

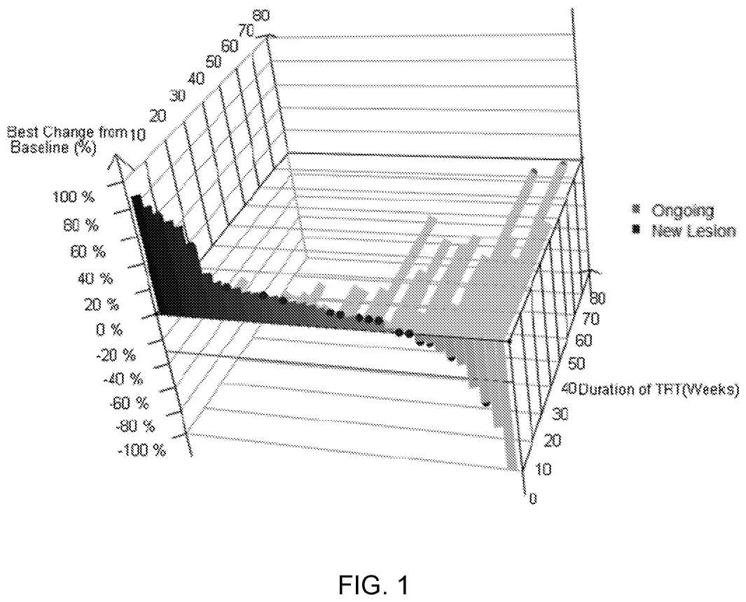

In third line therapy, MSS-CRC patients received durvalumab at 1500 mg every 4 weeks (Q4W)) in combination with monalizumab at 750 mg every 2 weeks (Q2W). The results showed that in the MSS-CRC expansion cohort (60% of patients having received at least 3 lines of prior therapy, n=39 evaluable for efficacy), there were 1 confirmed Complete Response, 2 confirmed Partial Response (PR) and 11 Stable Disease (SD). The Disease Control Rate (DCR) at 16 weeks was 31% and 18% at 24 weeks. Median OS thus far is encouraging of 10.6 months, which compares favourably to Lonsurf/TAS-102 median OS of 5.7 months (Mayer et al, (2015) N Engl J Med. 372:1909-1919) or against regorafenib reported median OS of 6.4 months (Grothey et al, (2013) Lancet, 381 (963): 303-312) in a similar population. Percent change in tumor size from baseline and duration of treatment in MSS-CRC expansion cohort are represented in FIG. 1.

In conclusion, the first-in-human combination of monalizumab plus durvalumab demonstrated a manageable toxicity profile. The data indicated that the monalizumab plus durvalumab combination could bring an improved benefit to patients with MSS-CRC, a population historically nonresponsive to PD-1/PD-L1 blockade.

### Example 4. Combination of a PD-1 Neutralizing Agent, a NKG2A Neutralizing Agent, Bevacizumab and Chemotherapeutic Regimen for Treatment of CRC

First Line patients (systemic therapy-naïve in the recurrent/metastatic setting) with advanced MSS-CRC received durvalumab (1500 mg every 4 weeks (Q4W)) in combination with monalizumab at 750 mg every 2 weeks (Q2W) plus a standard chemotherapy regimen of a modified FOLFOX regimen (mFOLFOX6) comprised of folinic acid (400 mg/m²), fluorouracil (400 mg/m² bolus followed by 2400 mg/m2 continuous IV infusion), and oxaliplatin (85 mg/m²) every 2 weeks (Q2W) in combination with bevacizumab (5 mg/kg) every 2 weeks (Q2W) according to institutional guidelines.

As of the data cut-off date of 25 Mar. 2019, 18 subjects have been enrolled and treated with monalizumab 750 mg Q2W, durvalumab 1500 mg Q4W, a modified FOLFOX regimen comprised of folinic acid, fluorouracil, and oxaliplatin [mFOLFOX6], and bevacizumab.

All of the 18 treated subjects experienced at least one Adverse Event (AE), and 14 subjects (77.8%) reported at least one event that was considered related to monalizumab and/or durvalumab. The most common (≥20%) treatment-emergent AEs were fatigue (12 subjects [66.7%]), nausea and neuropathy peripheral (10 subjects each [55.6%]), diarrhea (8 subjects [44.4%]), neutropenia, decreased appetite and temperature intolerance (7 subjects each [38.9%]), pyrexia and headache (6 subjects each [33.3%]), dysgeusia, oral pain and dizziness (5 subjects each [27.8%]), and epistaxis, amylase increased, lipase increased, blood bilirubin increased, aspartate aminotransferase increased, dyspnoea, constipation, and vomiting (4 subjects each [22.2%]). AEs considered by the investigators as related to monalizumab and/or durvalumab that occurred in >1 subject included fatigue (5 subjects [27.8%]), amylase increased and lipase increased (4 subjects each [22.2%]), aspartate aminotransferase increased, diarrhea and pyrexia (3 subjects each [16.7%]) and neutropenia, nausea, blood bilirubin increased, lymphocyte count decreased, decreased appetite, dysgeusia, headache, pruritus, and rash (2 subjects each [11.1%]). A total of 14 subjects (77.8%) experienced at least one Grade 3 or 4 AE; Grade 3 or 4 AEs that occurred in >1 subject included neutropenia and lipase increased (4 subjects each [22.2%]), lymphocyte count decreased (3 subjects [16.7%]), and small intestinal obstruction, alanine aminotransferase increased, aspartate aminotransferase increased, blood bilirubin increased, and neuropathy peripheral (2 subjects each [11.1%]). Nine subjects (50.0%) experienced at least one Grade 3 or 4 AE that was considered as related to monalizumab and/or durvalumab:lipase increased (4 subjects), lymphocyte count decreased (2 subjects), and neutropenia, amylase increased, alanine aminotransferase increased, aspartate aminotransferase increased, blood bilirubin increased, hyponatraemia, rash, and embolism (1 subject each).

One (1) subject experienced a Serious Adverse Event (SAE) that was considered related to monalizumab and/or durvalumab (SAE of embolism that was Grade 3 in severity and considered as related to monalizumab and bevacizumab). The event was reported as resolved and the subject was ongoing in the study as of the data cut-off date. One (1) subject discontinued treatment with monalizumab or durvalumab due to AE (AEs of alanine aminotransferase increased and aspartate aminotransferase increased that were both Grade 3 in severity and considered as related to monalizumab and durvalumab). The event of aspartate aminotransferase increased was reported as resolved and the event of alanine aminotransferase increased was downgraded to Grade 1 in severity and reported as ongoing. The subject completed end of study as of the data cut-off date. No subjects had Grade 5 (fatal) AEs as of the data cut-off date.

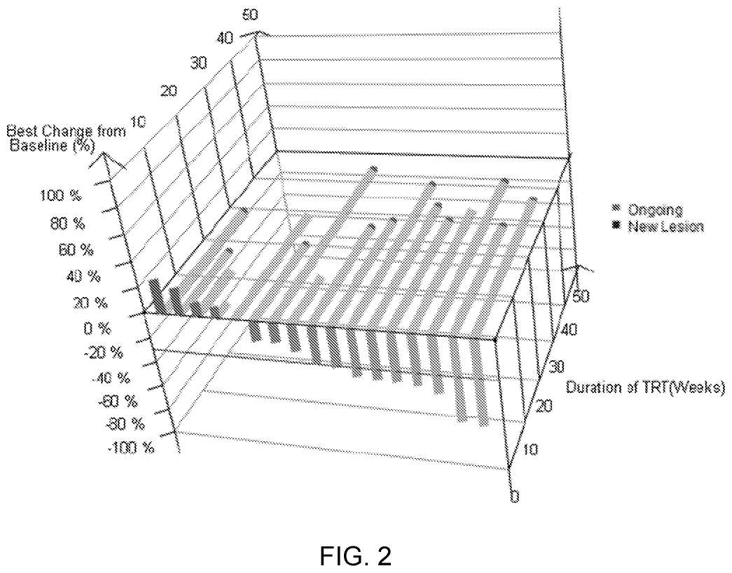

Of the 18 MSS-CRC dose-exploration subjects, 17 subjects were response-evaluable as of 26 Mar. 2019. Results are represented in FIG. 2. Seven subjects (41.2%) had a confirmed Partial Response (PR), and 2 subject's Stable Disease (SD) improved further to unconfirmed PR. Thirteen subjects (76.5%) maintained disease control (i.e., Complete Response (CR), PR, or SD) at 16 weeks.

The patients on study were then assessed again for safety and response rate. As of the cut off date of 29 Jul. 2019, the median follow-up was 10.0 months with a range of 1.6-14.2. Table 1. provides a summary of the safety data to date.

TABLE 1

| Safety parameter | MSS-CRC (N = 18) n (%) | |
|---|---|---|
| Any AEs | 18 | (100) |
| Grade 3/4 AEs | 14 | (77.8) |
| SAEs | 7 | (38.9) |
| Monalizumab-related AEs | 14 | (77.8) |
| Monalizumab-related SAEs | 1 | (5.6) |
| Durvalumab-related AEs | 15 | (88.3) |
| Durvalumab-related SAEs | 0 | |
| Chemotherapy-related AEs | 18 | (100) |
| Chemotherapy-related SAEs | 2 | (11.1) |
| Bevacizumab-related AEs | 10 | (55.6) |
| Bevacizumab-related SAEs | 2 | (11.1) |

Monalizumab-related adverse events (AEs) occurred in 14 patients (77.8%), most commonly fatigue (27.8%) and increased aspartate aminotransferase (16.7%). One patient (5.6%) had a serious monalizumab-related AE (SAE): Grade 3 embolism, which was also considered to be related to chemotherapy and bevacizumab.

Durvalumab-related AEs occurred in 15 patients (83.3%), most commonly fatigue (27.8%), increased amylase (22.2%), and increased lipase (22.2%). None had SAEs.

All patients had chemotherapy-related AEs, most commonly fatigue (55.6%), nausea (55.6%), and peripheral neuropathy (50.0%). Two patients (11.1%) had chemotherapy-related SAEs: Grade 3 embolism (the patient described above) and Grade 3 febrile neutropenia, which was also considered to be related to bevacizumab.

Bevacizumab-related AEs occurred in 10 patients (55.6%), most commonly epistaxis (16.7%), fatigue (16.7%), increased lipase (11.1%), and rash (11.1%). Two patients (11.1%) had bevacizumab-related SAEs: Grade 3 embolism and Grade 3 febrile neutropenia (the patients described above).

There were no Grade 5 AEs or DLTs.

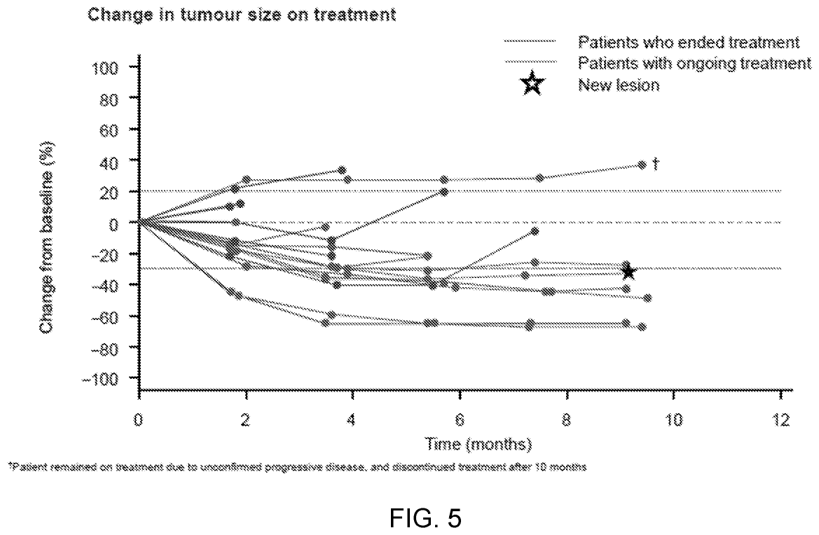

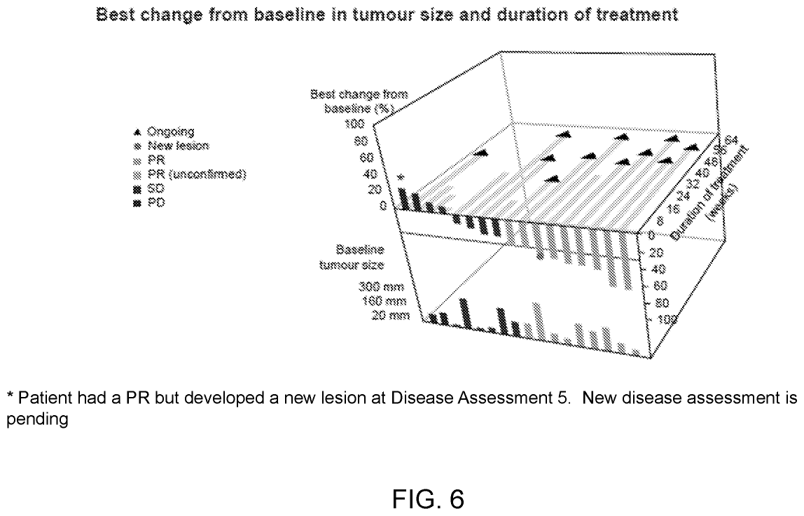

Clinical activity was also assessed. 17 patients were evaluable for response; 9 (52.9%) had partial responses (7 confirmed, 2 unconfirmed), 8 (47.1%) had stable disease, and 2 (11.8%) had progressive disease. There were no complete responses. Tumor size change and duration of treatment are shown in FIGS. 5 and 6. Median time to response was 15.4 weeks. Responses were durable (median not reached), ranging from 16.1 to 33.1 weeks. All but one of the responses were ongoing at the time of data analysis.

### Example 5. Enhanced Effect on Peripheral PD by FOLFOX and Bevacizumab on Monalizumab+Durvalumab Treatment in MSS-CRC

Circulating quantities of proliferating (Ki67+) NK and T cell populations were assessed using an analytically-validated flow cytometry assay on fresh whole blood (WB) specimens. In brief, WB collected in ACD-B anti-coagulant was incubated in two tubes with the following fluorochrome-labelled monoclonal antibodies: BV421-CD56, V500-CD45, PE-CD8, PerCP-Cy5.5-CD4, PE-Cy7 CD7, APC-CD3 and APC-H7-CD16 for 20 minutes on ice prior to erythrocyte lysis with FACS Lysing solution (BD Biosciences). Cells were subsequently washed in a 10% fetal bovine serum-containing PBS solution, fixed and permeabilized with Perm Buffer II (BD Biosciences). AF488-Ki67 or AF488-IgG (isotype control) were added, and cells were incubated for 20 minutes in the dark at room temperature prior to washing cells and analysis on a FACSCanto™ flow cytometer (BD Biosciences). Ki67+ T or NK cells were identified based on increased AF488 signal above that of the isotype control-stained cells.

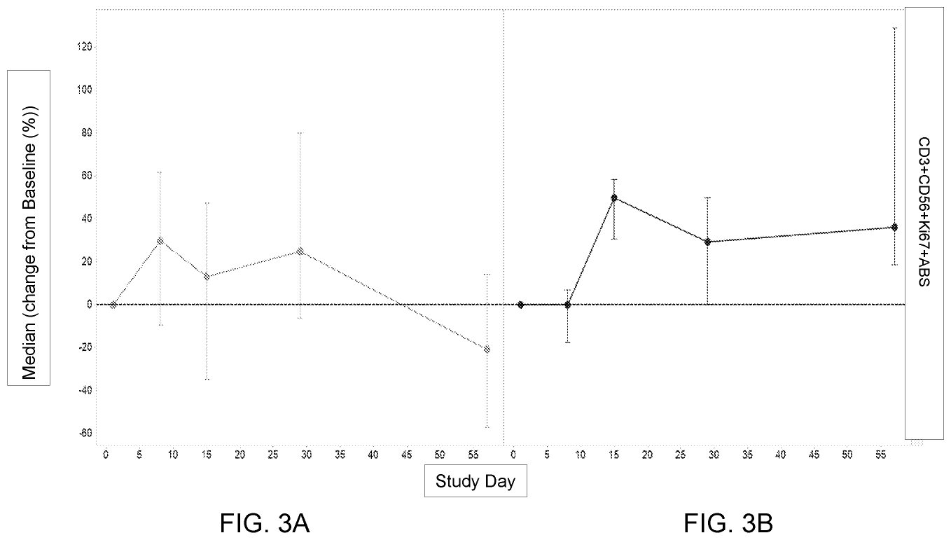

Median, baseline-normalized, proliferating CD16+CD56+ NK cells were elevated by 50% above baseline on day 15 in subjects receiving FOLFOX+bevacizumab+monalizumab+durvalumab (FIG. 3A and FIG. 3B). This represented an approximate 2-fold increase above peak median increases observed in MSS-CRC subjects receiving just monalizumab+durvalumab.

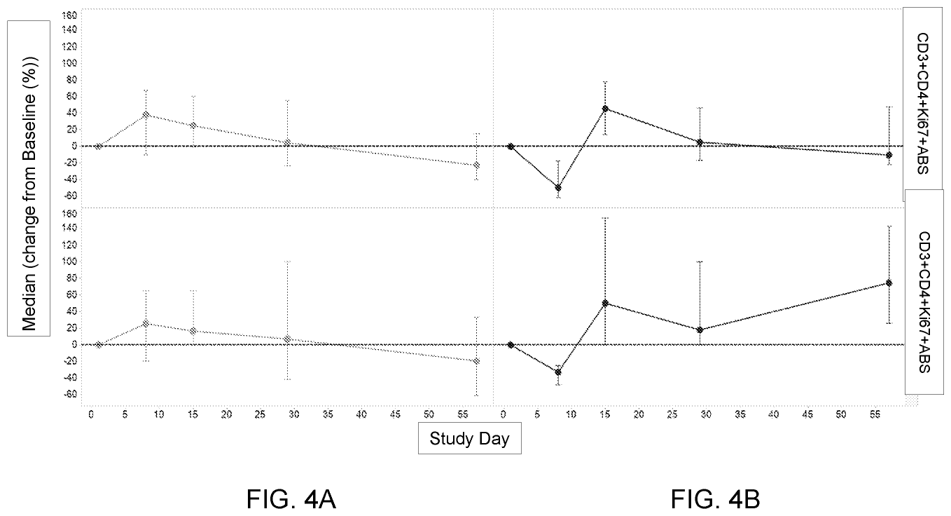

Elevations in proliferating CD4+ and CD8+ T cells of similar magnitudes were observed on day 15, but only median increases in baseline CD8+Ki67+ were greater than that observed in MSS-CRC patients receiving monalizumab+durvalumab (FIG. 4A and FIG. 4B). Day 8 decreases in median, baseline-normalized proliferating T cell populations were observed in subjects receiving FOLFOX+bevacizumab+monalizumab+durvalumab, and this likely reflects the immunosuppressive effects of oxaliplatin and fluorouracil (FIG. 4A and FIG. 4B).

### Example 6. Combination of a PD-1 Neutralizing Agent, a NKG2A Neutralizing Agent, Cetuximab and Chemotherapeutic Regimen for Treatment of MSS-CRC

Eligible patients (pts) had MSS-CRC (RAS/BRAF wild type with a left-sided colon primary tumor in the DMCC cohort) and ECOG PS 0-1. They received durvalumab 1500 mg Q4W, monalizumab 750 mg Q2W, modified FOLFOX6 Q2W and cetuximab up to 500 mg/m² Q2W for up to 3 yr. The primary endpoint was safety and tolerability; secondary endpoints included antitumor activity.

As of Aug. 26, 2019, 17 patients had received the combination therapy of durvalumab, monalizumab, chemotherapy, and cetuximab (DMCC). Monalizumab related adverse events (AEs) occurred in 47.1% of DMCC pts, including serious AEs (SAEs) in 11.8%. Durvalumab-related AEs occurred in 64.7% and 11.8% of the DMCC cohort had SAEs. 94.1% of DMCC pts had chemotherapy-related AEs, including SAEs in 11.8%. Biologic-related AEs occurred in 94.1%, including SAEs in 11.8%. There were no grade 5 AEs.

### Example 7. Follow-on Study of the Combination of a PD-1 Neutralizing Agent, a NKG2A Neutralizing Agent, Bevacizumab and Chemotherapeutic Regimen for First-Line Treatment of MSS-CRC

As described in Example 4, First Line patients (systemic therapy-naïve in the recurrent/metastatic setting) with advanced MSS-CRC received durvalumab (1500 mg every 4 weeks (Q4W)) in combination with monalizumab at 750 mg every 2 weeks (Q2W) plus a standard chemotherapy regimen of a modified FOLFOX regimen (mFOLFOX6) comprised of folinic acid (400 mg/m²), fluorouracil (400 mg/m² bolus followed by 2400 mg/m² continuous IV infusion), and oxaliplatin (85 mg/m²) every 2 weeks (Q2W) in combination with bevacizumab (5 mg/kg) every 2 weeks (Q2W) according to institutional guidelines. Treatment and assessment was continued to a cut off date of 24 Feb. 2020.

As of the data cut-off date of 24 Feb. 2020, 18 subjects have been enrolled and treated with monalizumab 750 mg Q2W, durvalumab 1500 mg Q4W, a modified FOLFOX regimen comprised of folinic acid, fluorouracil, and oxaliplatin [mFOLFOX6], and bevacizumab.

All of the 18 treated subjects experienced at least one Adverse Event (AE), and 15 subjects (83.3%) reported at least one event that was considered related to monalizumab and/or durvalumab. The most common (≥20%) treatment-emergent AEs were fatigue (13 subjects [72.2%]), nausea and neuropathy peripheral (11 subjects each [61.1%]), diarrhea and neutropenia (8 subjects each [44.4%]), decreased appetite and temperature intolerance (7 subjects each [38.9%]), pyrexia and headache (6 subjects each [33.3%]), and amylase increased, lipase increased (4 subjects each [22.2%]). AEs considered by the investigators as related to monalizumab and/or durvalumab that occurred in >1 subject included fatigue (5 subjects [27.8%]) and amylase increased and lipase increased (4 subjects each [22.2%]). A total of 16 subjects (88.9%) experienced at least one Grade 3 or 4 AE or Serious Adverse Effect (SAE); Grade 3 or 4 AEs that occurred in >1 subject included neutropenia (5 subjects [27.8%]), lipase increased (4 subjects [22.2%]), lymphocyte count decreased (3 subjects [16.7%]), and neuropathy peripheral and neutrophil count decreased (2 subjects each [11.1%]). Nine subjects (50.0%) experienced at least one Grade 3 or 4 AE that was considered as related to monalizumab and/or durvalumab; Grade 3 or 4 AEs that occurred in >1 subject included lipase increased and lymphocyte count decreased (2 subjects each [11.1%]). Three (3) subjects (16.7%) discontinued treatment with durvalumab due to AE. Two subjects (11.1%) discontinued treatment with monalizumab due to AE. No subjects had Grade 5 (fatal) AEs as of the data cut-off date.

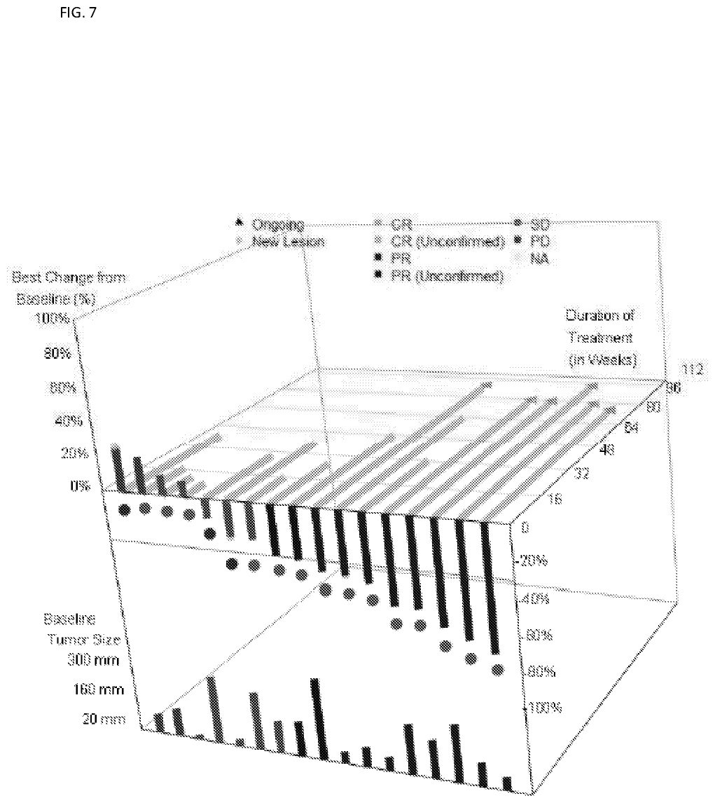

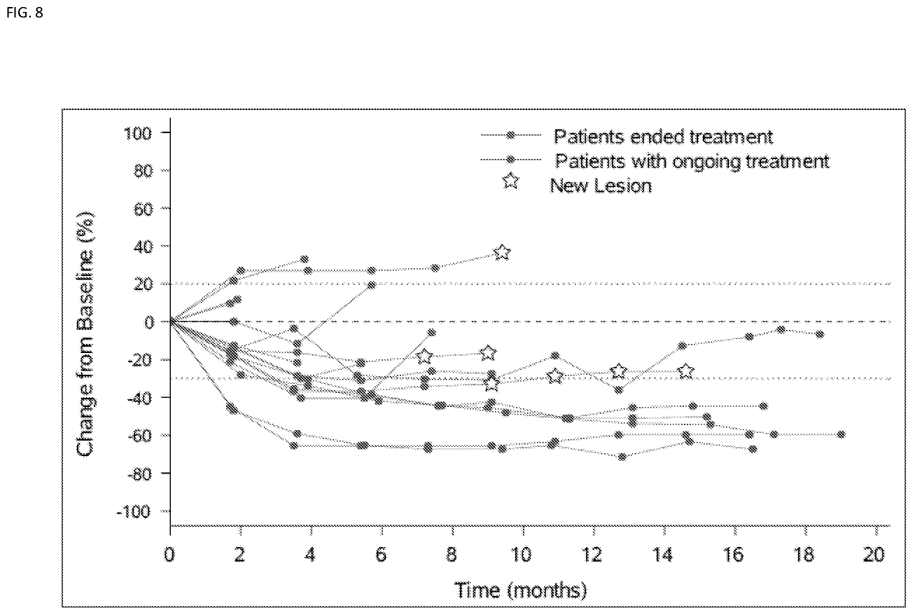

Clinical activity was also assessed, with the results shown in Table 2. 17 patients were evaluable for response; 10 (58.8%) had partial responses (8 confirmed, 2 unconfirmed), 5 (29.4%) had stable disease, and 2 (11.8%) had progressive disease. There were no complete responses. Tumor size change and duration of treatment are shown in FIGS. 7 and 8. The objective response rate (ORR) was 8 out of 17 subjects (47.1%). Fourteen subjects (82.4%) maintained disease control (i.e., Complete Response (CR), PR, or Stable Disease (SD)) at 16 weeks (DCR16).

TABLE 2
Results of monalizumab, durvalumab, mFOLFOX6
and bevacizumab combination treatment

| | Mona/Durva/FOLFOX6/Bev | |
|---|---|---|
| Efficacy | (N = 17) | |
| Complete Response (%) | 0 | (0) |
| Partial Response (%) | 8 | (47.1) |
| Static Disease (%) | 7 | (41.2) |
| Progressive Disease (%) | 2 | (11.8) |
| Objective Response Rate* (%) | 8/17 | (47.1) |
| DCR16 (%) (weeks) | 14 | (82.4) |

| | |
|---|---|
| Duration of Response (weeks) | 8 |
| median Duration of Follow-up | 16.9 |
| (months) | |
| PFS (months) | 9.1 |
| mOS (months) | NR |
| Details | 2 uPR (11.8%) - both have |
| | progressed. |

*Based on response-evaluable population (includes patients in as-treated population who have at least 1 post-baseline disease assessment or discontinued due to death or disease progression prior to the first scheduled disease assessment; NR = not reached.

### Example 8. Benchmarking of Combination of a PD-1 Neutralizing Agent, a NKG2A Neutralizing Agent, Bevacizumab and Chemotherapeutic Regimen for First-Line Treatment of MSS-CRC Against Previous Trials

The results of the subjects as reported in Example 7 above were then compared with the reported results of previous trials (Table 3). The subjects from Example 7 demonstrated a comparable objective response rate (ORR) to the subjects of the other trials. In particular, as reported in Example 7, an ORR of 47.1% was recorded for subjects receiving the monalizumab, durvalumab, mFOLFOX6 and bevacizumab combination treatment, whereas for subjects receiving a combination of FOLFIRI and bevacizumab according to the pivotal Hurwitz et al. trial an ORR of 44.8% was reported.

The results were then further interrogated based on the mutation status of the subjects (Table 4). RAS mutant subjects from Example 7 reported a higher partial response rate than any of the comparator trials for which data are available. For example, 5 of the 14 RAS mutant subjects from Example 7 exhibited a partial response (57.1%), whereas the highest partial response rate seen for RAS mutants in the comparator trials was 47.5% (the Stintzing et al. trial). The RAS mutant subjects from Example 7 demonstrated a comparable objective response rate (ORR) to the RAS/KRAS mutant subjects of the other trials.

### Example 9. Follow-on Study of the Combination of a PD-1 Neutralizing Agent, a NKG2A Neutralizing Agent, Cetuximab and Chemotherapeutic Regimen for Treatment of MSS-CRC

As described in Example 6, eligible First Line patients (pts) had MSS-CRC (RAS/BRAF wild type with a left-sided colon primary tumor in the DMCC cohort) and ECOG PS 0-1. They received durvalumab 1500 mg Q4W, monalizumab 750 mg Q2W, modified FOLFOX6 Q2W and cetuximab up to 500 mg/m² Q2W for up to 3 yr. The primary endpoint was safety and tolerability; secondary endpoints included antitumor activity. Treatment and assessment was continued to a cut off date of 24 Feb. 2020.

As of the data cut off date of 24 Feb. 2020, 17 subjects (94.4%) who had received the combination therapy of durvalumab, monalizumab, chemotherapy, and cetuximab (DMCC) had experienced one or more AE. Monalizumab/durvalumab related adverse events occurred in 15 subjects.

TABLE 3
Benchmarking of Mona/Durva/FOLFOX6/Bev against other reported trials

| | Mona/Durva/ | | | | | | FOLFIRI + | | |
|---|---|---|---|---|---|---|---|---|---|
| | FOLFOX6/Bev | | FOLFOXIRI + | | FOLFIRI + | FOLFOX/XELOX + | Bevacizumab | | FOLFOX + |
| | RAS/BRAF all | | Bevacizumab | | Bevacizumab | Bevacizumab | (FIRE-3) | | Bevacizumab |
| | comers | | All comers | | All comers | All comers | All comers | | All comers |
| Efficacy | (N = 17) | | (N = 252)¹ | | (N = 402***)² | (N = 699)³ | (N = 295)⁴ | | (N = 198)⁵ |
| CR | 0 | (0%) | 12 | (4.8%) | 3.7% | NA | 4 | (1%) | NA |
| PR | 8 | (47.1%) | 152 | (60.3%) | 41% | NA | 167 | (57%) | NA |
| SD | 7 | (41.2%) | 62 | (24.6%) | NA | NA | 85 | (29%) | NA |
| ORR | 8/17 | (47.1%) | 164 | (65.1%) | 44.8% | N/R | 171 | (58%) | 64% |

| | | | | | | | |
|---|---|---|---|---|---|---|---|
| DCR16 (%) | 14 | (82.4) | NA | NA | NA | NA | NA |
| (weeks) | | | | | | | |

| | | | | | | |
|---|---|---|---|---|---|---|
| PFS (months) | 9.1 | 12.1 | 10.6 | 9.4 | 10.3 | 10.7 |
| mOS (months) | NR | 31 | 20.3 | 21.3 | 25.0 | 30.1 |

***pivotal trial;
NA not available;
NR not reached
¹Loupakis et al. N Engl J Med. 2014; 371(17): 1609;
²Hurwitz, et al: N Engl J Med 350: 2335-2342, 2004;
³Saltz, et al. J Clin Oncol. 2008 Apr. 20; 26(12): 2013-9;
⁴Heinemann et al. Lancet Oncol. 2014 September; 15(10): 1065-75;
⁵Yamazaki et al. Ann Oncol. 2016 August; 27(8): 1539-46.

TABLE 4
Benchmarking of Mona/Durva/FOLFOX6/Bev according to RAS/KRAS status against other reported trials

| | | | | | | | | | FOLFOX/FOLFIRI + | | | | | | | |
|---|---|---|---|---|---|---|---|---|---|---|---|---|---|---|---|---|
| | | | | | FOLFIRI + | | | | Bevacizumab | | FOLFIRI + | | | | CAPEOX + | |
| | Mona/Durva/ | | | | Bevacizumab | | | | (CALGB/SWOG | | Bevacizumab | | | | Bevacizumab | |
| | FOLFOX6/Bev | | | | (AIO KRK-0306)¹ | | | | 80405)² | | (AVF2107)³ | | | | (CAIRO-2)⁴ | |
| | (N = 17) | | | | (N = 316) | | | | (N = 532) | | (N = 129) | | | | (N = 264) | |
| | RAS | | RAS | | RAS | | RAS | | RAS | RAS | KRAS | | KRAS | | KRAS | KRAS |
| | wild-type | | mutant | | wild-type | | mutant | | wild-type | mutant | wild-type | | mutant | | wild-type | mutant |
| Efficacy | (N = 3) ^{§} | | (N = 14) | | (N = 257) | | (N = 59) | | (N = 217) | (N = 98) | (N= 85) | | (N = 44) | | (N = 156) | (N = 108) |
| CR | 0 | (0%) | 0 | (0%) | 2 | (0.8%) | 0 | | NA | 0 | 3 | (3.5%) | 3 | (6.8%) | NA | NA |
| PR | 0 | (0%) | 8 | (57.1%) | 141 | (54.9%) | 28 | (47.5%) | NA | NA | 48 | (56.5%) | 16 | (36.4%) | NA | NA |
| SD | 2 | (66.6%) | 5 | (35.7%) | 84 | (32.7%) | 21 | (35.6%) | NA | NA | NA | | NA | | NA | NA |
| ORR | 0 | (0%) | 8 | (57.1%) | 143 | (55.6%) | 28 | (47.5%) | NA | NA | 51 | (60%) | 19 | (43.2%) | 50% | 59.2% |

| | | | | | | | | | | |
|---|---|---|---|---|---|---|---|---|---|---|
| PFS | NR | NR | NA | 9.7 | 11.1 | 12 | 13.5 | 9.3 | 10.6 | 12.5 |
| (months) | | | | | | | | | | |
| Median OS | NR | NR | NA | 20.1 | 33.3 | 28.1 | 27.7 | 19.9 | 22.4 | 24.9 |
| (months) | | | | | | | | | | |

NA not available;
NR not reached
¹Stintzing et al. European Journal of Cancer 79: (2017)50-60;
²Innocenti et al. J Clin Oncol 37: 1217-1227;
³Hurwitz et al. The Oncologist 2009; 14: 22-28;
⁴Tol et al. N Engl J Med 2019; 381: 1644-1652.

(83.3%), with increased lipase and increased amylase being the most common (7 subjects (38.9%) for each). At least one grade 3/4 AE, or SAEs were observed in 17 subjects (94.4%), with increased lipase being the most common (6 subjects, 33.3%). 11 subjects (61.1%) experienced at least one treatment-related grade 3/4 AE, of these, increased lipase was most common (4 subjects, 22.2%). There were no grade 5 AEs and no fatal AEs. Two (2) subjects (11.1%) discontinued treatment with durvalumab due to AE. Two subjects (11.1%) discontinued treatment with monalizumab due to AE.

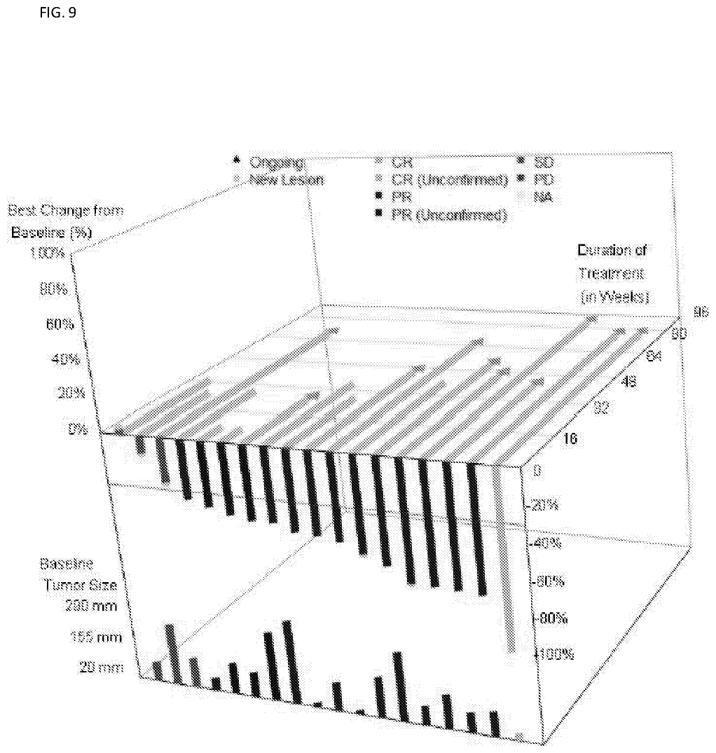

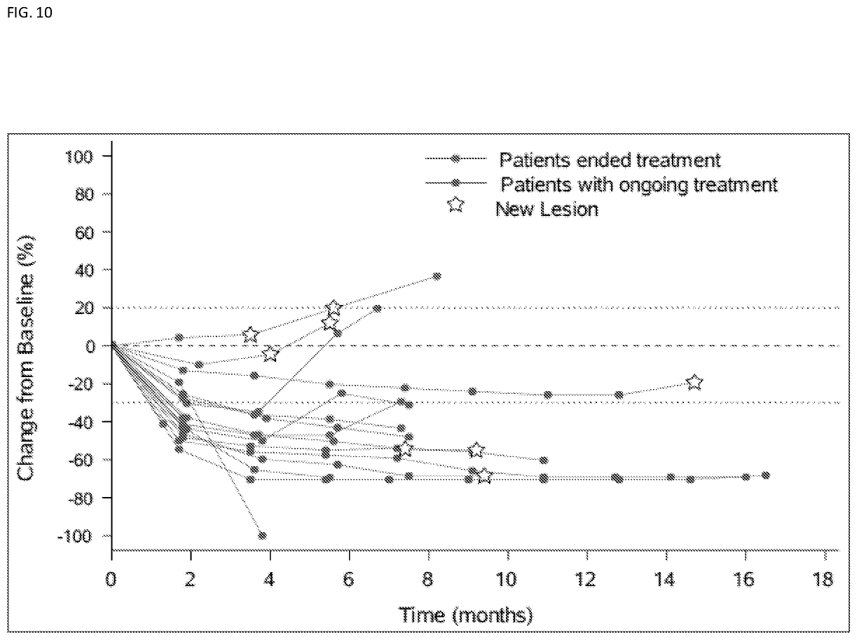

Clinical activity was also assessed, with the results shown in Table 5. 18 patients were evaluable for response; 11 (61.1%) had confirmed partial responses (3 further unconfirmed PR), 6 (33.3%) had stable disease, and none had progressive disease. There were no confirmed complete responses. Tumor size change and duration of treatment are shown in FIGS. 9 and 10. The objective response rate (ORR) was 11 out of 18 subjects (61.1%). Fifteen subjects (83.3%) maintained disease control (i.e., Complete Response (CR), PR, or Stable Disease (SD)) at 16 weeks (DCR16).

TABLE 5
Results of monalizumab, durvalumab, mFOLFOX6
and cetuximab combination treatment

| | Mona/Durva/FOLFOX6/Cetux | |
|---|---|---|
| Efficacy | (N = 18) | |
| CR (%) | 0 | (0) |
| PR (%) | 11 | (61.1) |
| SD (%) | 6 | (33.3) |
| PD (%) | 0/18 | (0) |
| ORR* (%) | 11/18 | (61.1) |
| DCR16 (%) (weeks) | 15 | (83.3) |

| | |
|---|---|
| Duration of Response | 11 |
| (weeks) | |
| median Duration of Follow- | 12.8 |
| up (months) | |
| PFS (months) | NR |
| mOS (months) | NR |
| Details | 1 ongoing uCR (5.6%), |
| | 3 uPR (16.7%) - 1 ongoing, 1 |
| | progressed, 1 withdrew consent |

*Based on response-evaluable population (includes patients in as-treated population who have at least 1 post-baseline disease assessment or discontinued due to death or disease progression prior to the first scheduled disease assessment; NR = not reached.

### Example 10. Benchmarking of Combination of a Combination of a PD-1 Neutralizing Agent, a NKG2A Neutralizing Agent, Cetuximab and Chemotherapeutic Regimen for Treatment of MSS-CRC Against Previous Trials

The results of the subjects as reported in Example 9 above were then compared with the reported results of previous trials (Table 6). The subjects from Example 9 demonstrated a comparable objective response rate (ORR) to the subjects of the other trials. In particular, as reported in Example 9, an ORR of 61.1% was recorded for subjects receiving the monalizumab, durvalumab, mFOLFOX6 and cetuximab combination treatment, which is higher than the ORR for all but one of the comparator trials (FOLFIRI and panitumumab gave a reported ORR of 87.3%).

TABLE 6
Benchmarking of Mona/Durva/FOLFOX6/Cetux against other reported trials

| | | | Cetuximab + FOLFIRI | | | | Panitumumab + FOLFOX4 | | | | | |
|---|---|---|---|---|---|---|---|---|---|---|---|---|
| | Mona/Durva/ | | First Line¹ | | | | First Line² | | | | Panitumumab + | |
| | FOLFOX6/Cetux | | (N = 530) | | | | (N = 532) | | | | FOLFOXIRI³ | |
| | (N = 18) | | KRAS | | KRAS | | KRAS | | KRAS | | (N = 63) | |
| | RAS/BRAF wt + | | wild-type | | mutant | | wild-type | | mutant | | RAS | |
| Efficacy | left-sided | | (N = 316) | | (N = 214) | | (N = 317) | | (N = 215) | | wild-type | |
| CR (%) | 0 | | 3 | (0.9) | 0 | | 1 | (0.3) | 0 | | 0 | |
| PR (%) | 11 | (61.1) | 178 | (56.3) | 67 | (31.3) | 180 | (57) | 86 | (40) | 55 | (87.3) |
| SD (%) | 6 | (33.3) | 100 | (31.6) | 101 | (47.2) | 91 | (29) | 80 | (37) | 5 | (7.9) |

| | | | | | | | | | | |
|---|---|---|---|---|---|---|---|---|---|---|
| ORR | 11 | (61.1) | 57.3% | 31.3% | 181 | (57.1%) | 86 | (40.0%) | 55 | (87.3%) |

| | | | | | | | |
|---|---|---|---|---|---|---|---|
| DCR16 (%) | 15 | (83.3) | NA | NA | NA | NA | NA |
| (weeks) | | | | | | | |

| | | | | | | |
|---|---|---|---|---|---|---|
| PFS | NR | 9.9 | 7.4 | 10.0 | 7.4 | 9.7 |
| (months) | | | | | | |
| mOS | NR | 23.5 | 16.2 | 23.9 | 15.5 | 35.7 |
| (months) | | | | | | |

DCR16 = CR + PR + SD ≥ 16 weeks;
NA not available;
NR not reached
¹Van Cutsem et al J Clin Oncol. 2011; 29(15): 2011-2019;
²Douillard et al Annals of Oncology. 2014; 25: 1346-1355;
³Modest et al J Clin Oncol. 2019 Oct. 14: JCO1901340

1-100. (canceled)

101. A method of reducing or inhibiting colorectal tumor growth in a subject in need thereof, comprising administering to the subject a therapeutically effective amount of each of (i) monalizumab, (ii) durvalumab, (iii) a FOLFOX agent, and (iv) bevacizumab.

102. The method according to claim 101, wherein the subject has a colorectal tumor that is not microsatellite Instability-High (MSI-H) and/or not DNA mismatch repair (MMR) defective.

103. The method according to claim 101, wherein the subject has a colorectal tumor that does not have microsatellite instability detected in two or more microsatellite markers, wherein the subject has a colorectal tumor that has no alteration detected in two or more of the microsatellite markers selected from the group consisting of BAT-25, BAT-26, NR-21, NR-24, and MON027.

104. The method according to claim 101, wherein the subject has a colorectal tumor that does not have an alteration in expression of a DNA mismatch repair (MMR) protein, wherein the subject has a colorectal tumor that does not have decreased or absence of expression of at least one MMR protein selected from MSH2, MLH1, MSH6 and PMS2.

105. The method according to claim 101, wherein the subject has a colorectal tumor that is microsatellite stable (MSS).

106. The method according to claim 101, wherein the colorectal tumor is an advanced recurrent or a metastatic colorectal tumor.

107. The method according to claim 101, wherein the subject has a microsatellite stable-colorectal cancer (MSS-CRC).

108. The method according to claim 101, wherein the FOLFOX agent comprises oxaliplatin, 5-fluorouracil and leucovorin.

109. The method according to claim 101, wherein monalizumab, durvalumab, the FOLFOX agent, and bevacizumab are administered simultaneously, separately, or sequentially.

110. The method according to claim 101, wherein monalizumab, durvalumab, the FOLFOX agent, and bevacizumab are formulated for separate administration and are administered concurrently or sequentially.

111. The method according to claim 101, wherein the FOLFOX agent comprises folinic acid, fluorouracil, and oxaliplatin.

112. The method according to claim 101, wherein monalizumab is administered at a fixed dose of 750 mg every 2 weeks, durvalumab is administered at a fixed dose of 1500 mg/kg every 4 weeks, the FOLFOX agent comprises folinic acid administered at a fixed dose of 400 mg/m², fluorouracil administered at a fixed dose of 400 mg/m² bolus followed by 2400 mg/m² continuous IV infusion, and oxaliplatin administered at a fixed dose of 85 mg/m² every 2 weeks, and bevacizumab is administered at a fixed dose of 5 mg/kg every 2 weeks.

113. A method of treating colorectal cancer in a subject in need thereof, comprising administering to the subject a therapeutically effective amount of each of (i) monalizumab, (ii) durvalumab, (iii) a FOLFOX agent, and (iv) bevacizumab.

114. The method according to claim 113, wherein the subject has a colorectal cancer that is not microsatellite Instability-High (MSI-H) and/or not DNA mismatch repair (MMR) defective.

115. The method according to claim 113, wherein the subject has a colorectal cancer that does not have microsatellite instability detected in two or more microsatellite markers, wherein the subject has a colorectal cancer that has no alteration detected in two or more of the microsatellite markers selected from the group consisting of BAT-25, BAT-26, NR-21, NR-24, and MON027.

116. The method according to claim 113, wherein the subject has a colorectal cancer that does not have an alteration in expression of a DNA mismatch repair (MMR) protein, wherein the subject has a colorectal cancer that does not have decreased or absence of expression of at least one MMR protein selected from MSH2, MLH1, MSH6 and PMS2.

117. The method according to claim 113, wherein the subject has a colorectal cancer that is microsatellite stable (MSS).

118. The method according to claim 113, wherein the colorectal cancer is an advanced recurrent or a metastatic colorectal cancer.

119. The method according to claim 113, wherein the subject has a microsatellite stable-colorectal cancer (MSS-CRC).

120. The method according to claim 113, wherein the FOLFOX agent comprises oxaliplatin, 5-fluorouracil and leucovorin.

121. The method according to claim 113, wherein monalizumab, durvalumab, the FOLFOX agent, and bevacizumab are administered simultaneously, separately, or sequentially.

122. The method according to claim 113, wherein monalizumab, durvalumab, the FOLFOX agent, and bevacizumab are formulated for separate administration and are administered concurrently or sequentially.

123. The method according to claim 113, wherein the FOLFOX agent comprises folinic acid, fluorouracil, and oxaliplatin.

124. The method according to claim 113, wherein monalizumab is administered at a fixed dose of 750 mg every 2 weeks, durvalumab is administered at a fixed dose of 1500 mg/kg every 4 weeks, the FOLFOX agent comprises folinic acid administered at a fixed dose of 400 mg/m², fluorouracil administered at a fixed dose of 400 mg/m² bolus followed by 2400 mg/m² continuous IV infusion, and oxaliplatin administered at a fixed dose of 85 mg/m² every 2 weeks, and bevacizumab is administered at a fixed dose of 5 mg/kg every 2 weeks.
